SEQUENCE LISTING (12 sequences)

SEQ ID NO: 1 - protein, 117 aa, Artificial Sequence [sequence omitted]

SEQ ID NO: 2 - protein, 108 aa, Artificial Sequence [sequence omitted]

SEQ ID NO: 3 - protein, 5 aa, Artificial Sequence
  HCDR1
NYWIA

SEQ ID NO: 4 - protein, 17 aa, Artificial Sequence
  HCDR2
IIYPGDSDIRYSPSFQG

SEQ ID NO: 5 - protein, 8 aa, Artificial Sequence
  HCDR3
HDIEGFDY

SEQ ID NO: 6 - protein, 12 aa, Artificial Sequence
  LCDR1
RASQSVSSSFFA

SEQ ID NO: 7 - protein, 7 aa, Artificial Sequence
  LCDR2
GASSRAT

SEQ ID NO: 8 - protein, 9 aa, Artificial Sequence
  LCDR3
QQYDSSAIT

SEQ ID NO: 9 - protein, 107 aa, Artificial Sequence [sequence omitted]

SEQ ID NO: 10 - protein, 330 aa, Artificial Sequence
  Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 447 aa, Artificial Sequence
  Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial Sequence
  Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0026015 A1
(43) Pub. Date: Jan. 25, 2024

(54) TREATMENT OF FLARES IN LUPUS

(71) Applicant: ASTRAZENECA AB, SODERTALJE (SE)

(72) Inventors: RUBANA KALYANI, GAITHERSBURG, MD (US); GABRIEL ABREU, SODERTALJE (SE); RAJENDRA TUMMALA, WILMINGTON, DE (US); RICHARD FURIE, MANHASSET, NY (US); ERIC MORAND, CLAYTON (AU); ANCA ASKANASE, NEW YORK, NY (US); ED VITAL, LEEDS (GB); KENNETH KALUNIAN, LA JOLLA, CA (US); CATHARINA LINDHOLM, SODERTALJE (SE); EMMANUELLE MAHO, CAMBRIDGE (GB); CHRISTI KLEOUDIS, WILMINGTON, DE (US)

(21) Appl. No.: 18/248,280

(22) PCT Filed: Oct. 7, 2021
(86) PCT No.: PCT/EP2021/077702

§ 371 Date: Apr. 7, 2023

Related U.S. Application Data
(60) Provisional application No. 63/089,355, filed on Oct. 8, 2020.
(60) Provisional application No. 63/178,748, filed on Apr. 23, 2021.

(51) Int. Cl. C07K 16/28; A61K 9/00; A61P 13/12; A61P 19/02; A61P 37/06

(52) U.S. Cl. CPC C07K 16/2866; A61K 9/0019; A61P 13/12; A61P 19/02; A61P 37/06

(57) ABSTRACT

The disclosure relates to methods and compositions for the treatment of Systemic Lupus Erythematosus (SLE). The disclosure particular relates to the treatment of flares in SLE across multiple organ domains.

### 1 BACKGROUND

### 1.1 Systemic Lupus Erythematosus (SLE)

Systemic lupus erythematosus (SLE) is a chronic, multisystemic, disabling autoimmune rheumatic disease of unknown aetiology. There is substantial unmet medical need in the treatment of SLE, particularly in subjects with moderate or severe disease. Long-term prognosis remains poor for many subjects.

A significant problem associated with the treatment of SLE, is the heterogeneous clinical manifestations of SLE¹. Any organ may be affected in SLE, with the skin, joints, and kidneys being the most commonly involved²⁻⁴. Incomplete disease control leads to progressive organ damage, poor quality of life, and increased mortality, with approximately half of all patients with SLE developing organ damage within 10 years of diagnosis^{5,6}. There remains the need for a medical intervention that improves SLE disease activity across multiple systems.

Clinical manifestations of SLE include, but are not limited to, constitutional symptoms, alopecia, rashes, serositis, arthritis, nephritis, vasculitis, lymphadenopathy, splenomegaly, haemolytic anaemia, cognitive dysfunction and other nervous system involvement. Increased hospitalisations and side effects of medications including chronic oral corticosteroids (OCS) and other immunosuppressive treatments add to disease burden in SLE⁷-9.

All of the therapies currently used for the treatment of SLE have well known adverse effect profiles and there is a medical need to identify new targeted therapies, particularly agents that may reduce the requirement for corticosteroids and cytotoxic agents. There has been only 1 new treatment (belimumab) for SLE approved by the US Food and Drug Administration (FDA) and European Medicines Agency (EMA) in the approximately 50 years since hydroxychloroquine was approved for use in discoid lupus and SLE. However, belimumab is not approved everywhere, and the uptake has been modest. Many agents currently used to treat SLE, such as azathioprine, cyclophosphamide, and mycophenolate mofetil/mycophenolic acid, have not been approved for the disease. Furthermore, these drugs all have well-documented safety issues and are not effective in all patients for all manifestations of lupus. Antimalarial agents (e.g. hydroxychloroquine) and corticosteroids may be used to control arthralgia, arthritis, and rashes. Other treatments include nonsteroidal anti-inflammatory drugs (NSAIDs); analgesics for fever, arthralgia, and arthritis; and topical sunscreens to minimise photosensitivity. It is often difficult to taper subjects with moderate or severe disease completely off corticosteroids, which cause long-term morbidity and may contribute to early cardiovascular mortality^{8,10}. Even small daily doses of 5 to 10 mg prednisone used long-term carry increased risks of side effects such as cataracts, osteoporosis, and coronary artery disease⁸.

### 1.2 the Challenge of Finding a Treatment for SLE

The clinical development of a new drug is a lengthy and costly process with low odds of success. For molecules that enter clinical development, less than 10% will eventually be approved by health regulatory authorities¹¹. Furthermore, the early clinical development of biotherapeutics is much lengthier than for small molecules.

Phase II trials are conducted in a small number of volunteers who have the disease of interest. They are designed to test safety, pharmacokinetics, and pharmacodynamics. A phase II trial may offer preliminary evidence of drug efficacy. However, the small number of participants and primary safety concerns within a phase II trial usually limit its power to establish efficacy. A Phase III trial is required to demonstrate the efficacy and safety of a clinical candidate. Critically, many clinical candidates that have shown promise at Phase II fail at Phase III. More than 90% of novel therapeutics entering Phase I trials fail during clinical development, primarily because of failure in efficacy or safety. The probability of success at phase III, following successful Phase II, is less than 50%¹².

The process of drug development is particularly difficult for SLE. This is because SLE is an especially complex and poorly understood disease. Not only is our understanding of the genetics of SLE rudimentary, but our insight into pathogenesis of most of the clinical manifestations are still relatively limited compared to other disease.

The complexity of SLE presents those wishing to develop new therapeutics with the problem of a patient population with extensive inhomogeneity¹³. This makes protocol design for clinical trials in SLE even more difficult, for example, as regards to the choice of inclusion criteria and primary and secondary endpoints. It is further difficult to predict the disease course in each patient. This inevitably increases the background noise that reduces the statistical power of a trial. A high placebo response rate limits the range in which the tested new drug can show an efficacy signal, making clinical trials even more difficult to conduct and interpret.

The difficulty in developing effective therapeutics for SLE leads to an even higher failure rate of therapeutics in this area in clinical trials, compared to therapeutics for other indications. The development of novel therapeutics for the treatment of SLE has thus proved extremely difficult. There are many examples of clinical candidates that showed promise at Phase II but failed to show efficacy and/or safety in subsequent Phase or Phase III trials:

### 1.3 Tabalumab

Tabalumab (LY2127399) is a human IgG4 monoclonal antibody that binds both soluble and membrane-bound B-cell activating factor (BAFF). The efficacy and safety of tabalumab was assessed in two 52-week, phase III, multicentre randomized, double-blind, placebo-controlled trial in patients with moderate-to-severe SLE (ILLUMINATE-1 and ILLUMINATE-2). The primary endpoint was proportion of patients achieving SLE Responder Index 5 (SRI-5) response at week 52. In ILLUMINATE-1 (NCT01196091), the primary endpoint was not met. Key secondary efficacy endpoints (OCS sparing, time to severe flare, worst fatigue in the last 24 hours) also did not achieve statistical significance, despite pharmacodynamic evidence of tabalumab biological activity (significant decreases in anti-dsDNA, total B-cells, and immunoglobulins)¹⁴. The primary endpoint was met in ILLUMINATE-2 (NCT01205438) in the higher dose group (tabalumab 120 mg every 2 weeks). However, no secondary endpoints were met, including OCS sparing¹⁵. Following ILLUMINATE-1 and ILLUMINATE-2, tabalumab development was suspended given the small effect size and inability to meet other important clinical endpoints.

### 1.4 Blisibimod

Blisibimod is a fusion protein composed of four BAFF-binding domains fused to the N-terminal Fc fragment of human IgG1 Ig. Blisibimod for the treatment of SLE had promising Phase II results but was unsuccessful in Phase III. In a phase 2 double-blind, randomized, placebo-controlled clinical trial (PEARL-SC), patients with serologically active SLE and SELENA-SLEDAI score ≥6 points were randomized to 3 different doses of blisibimod or placebo (NCT01162681). At week 24, the highest dose group (200 mg once weekly) had a significantly higher SRI-5 response rate than the placebo group¹⁶. However, in a subsequent placebo-controlled, phase III randomized, double-blind study (CHABLIS-SC1) conducted on seropositive SLE patients with persistent high disease activity (SELENA-SLEDAI ≥10 points) the primary endpoint (SRI-6) was not met (NCT01395745). The secondary end points (SRI-4 and SRI-8) were also not reached¹⁷.

### 1.5 Atacicept

Atacicept (TACI-Ig) is a fully human recombinant fusion protein that neutralizes both BAFF and APRIL. The efficacy of atacicept for the treatment of SLE was evaluated in two phase II/III placebo randomized controlled trials (APRIL-LN and APRIL-SLE). The APRIL-LN trial compared renal response to atacicept versus placebo plus standard of care (newly initiated MMF and glucocorticoids) in patients with SLE nephritis. The trial was discontinued after serious adverse events were reported. In APRIL-SLE the primary end point, defined as a significantly decreased proportion of patients who developed a new flare from BILAG A or BILAG B domain scores, was not met in the lower dose (75 mg) arm (NCT00624338). Treatment of patients with the higher dose (150 mg) arm was discontinued due to serious AEs¹⁸.

### 1.6 Abetimus

Abetimus (LJP 394) comprises four synthetic oligodeoxynucleotides attached to a triethyleneglycol backbone, where more than 97% of these oligonucleotides are derived from dsDNA. The drug was designed to neutralize anti-dsDNA antibodies. In a double-blind, placebo-controlled study in SLE patients, treatment with UP 394 in patients with high-affinity antibodies to its DNA epitope prolonged the time to renal flare, decreased the number of renal flares¹⁹. However, in a subsequent Phase III trial (NCT00089804) using higher doses of abetimus, with a primary endpoint of time to renal flare, study and further drug development was discontinued when interim analysis failed to show efficacy²⁰.

### 1.7 Rituximab

Rituximab is a chimeric anti-CD20 monoclonal antibody. Rituximab is an effective treatment in a number of autoimmune diseases, including rheumatoid arthritis and ANCA vasculitis. A small number of uncontrolled trials in lupus nephritis suggested that rituximab could also be potentially effective in patients with lupus nephritis. Efficacy and safety of rituximab was assessed in a randomized, double-blind, placebo-controlled phase III trial in patients with lupus nephritis treated concomitantly with mycophenolate mofetil (MMF) and corticosteroids (LUNAR) (NCT00282347). Rituximab therapy did not improve clinical outcomes after 1 year of treatment²¹. The efficacy and safety of rituximab in patients with moderate to severe SLE was evaluated in a multicentre placebo randomized controlled phase II/III trial (EXPLORER). The study randomized patients with baseline active SLE (defined as ≥1 new BILAG A scores or ≥2 BILAG B scores) to rituximab or placebo. The primary endpoint was the proportion of rituximab versus placebo-treated patients achieving a complete clinical response (CCR), partial clinical response (PCR), or no response at week 52. The primary endpoint was not met, with similar rates of complete and partial responses in rituximab and placebo arms at 52 weeks. Differences in time to first moderate or severe flare and change in HRQOL were also not significant²².

### 1.8 Abatacept

Abatacept is a CTLA-4 fusion protein that binds to CD80/86 on the surface of antigen presenting cells and blocks signalling through CD-28 required for T-cell activation. In preclinical studies abatacept was demonstrated to have immunomodulatory activity in the NZB/NZW murine model of lupu²³. Abatacept for treatment of non-renal SLE was been evaluated in a phase IIb, randomized, double-blind, placebo-controlled trial²⁴ (NCT00119678). The primary end point was the proportion of patients with new flare (adjudicated) according to a score of A/B on the British Isles Lupus Assessment Group (BILAG) index after the start of the steroid taper. The primary and secondary end points were not met.

### 1.9 Epratuzumab

Epratuzumab is a monoclonal antibody that modulates B-cell activity by binding CD22 on the surface of mature B-cells. Epratuzumab initially demonstrated efficacy in treating SLE at phase II trial but this was not confirmed in a follow-up second phase IIb trial or the subsequent phase III trial. Two phase IIb trials assessed the efficacy of epratuzumab with a BILAG-based primary endpoint in patients with moderate-to-severe SLE (ALLEVIATE 1 and 2). A trend towards clinical efficacy was observed and the primary end point was met by more patients treated with epratuzumab than placebo. Epratuzumab treatment also led to improvements in Health-related quality of life (HRQOL) and mean glucocorticoid dose²⁵. In another phase IIb trial (EMBLEM), patients with moderate-to-severe SLE were randomized to one of five epratuzumab doses or placebo. BICLA response at 12 weeks, the primary endpoint, was greater with all doses of epratuzumab than placebo, but the effect was not statistically significant. In the subsequent multicentre phase III trials EMBODY 1 and EMBODY 2, patients with moderate-to-severe SLE, the primary efficacy endpoint, BICLA response at 48 weeks, was not met. No significant differences were seen in secondary endpoints such as total SLEDAI-2K score, PGA, or mean glucocorticoid dose²⁶.

### 1.10 PF-04236921

PF-04236921 is a monoclonal antibody that binds soluble IL-6, a cytokine that is elevated in SLE patients. The efficacy of PF-0436921 was evaluated in a phase II RCT of patients with active SLE (BUTTERFLY) (NCT01405196). Patients were randomized to receive either subcutaneous PF-04236921 10 mg, 50 mg, or 200 mg or placebo every 8 weeks; the 200 mg dose arm was discontinued early because of 3 deaths. The primary efficacy endpoint was SRI-4 response at 24 weeks, with BICLA as a secondary endpoint. The primary endpoint was not met²⁷.

### 1.11 Type I IFN and Anifrolumab

Anifrolumab (MEDI-546) is a human immunoglobulin G1 kappa (IgG1K) monoclonal antibody (mAb) directed against subunit 1 of the type I interferon receptor (IFNAR1). It is composed of 2 identical light chains and 2 identical heavy chains, with an overall molecular weight of approximately 148 kDa. Anifrolumab inhibits binding of type I IFN to type I interferon receptor (IFNAR) and inhibits the biologic activity of all type I IFNs.

Type I interferons (IFNs) are cytokines that have been implicated in SLE pathogenesis based on the finding of increased IFN-stimulated gene expression in most patients with SLE. In the phase 3 TULIP-2 trial of anifrolumab in patients with moderate to severe SLE, treatment response (assessed using British Isles Lupus Assessment Group [BILAG]-based Composite Lupus Assessment [BICLA]) was achieved by significantly more patients receiving anifrolumab compared with placebo at Week 52²⁸. Similar results with this composite endpoint were observed in the phase 2 MUSE and phase 3 TULIP-1 trials^{29,30}. Importantly, composite endpoints used in SLE trials, such as BICLA and the SLE responder index (SRI), dichotomize changes in disease activity across different organ domains into a binary responder versus nonresponder result. While helpful for definitive demonstration of efficacy, this approach limits the ability to interpret treatment efficacy across the many organ domains that potentially affect patients with SLE.

### 1.12 CONCLUSION

There is a huge unmet need for an SLE therapy with a better efficacy and safety profile the currently available therapies^{31,32}. As described above, a large number and broad range of different biologics have been proposed and subjected to clinical trials, but these trials have failed to meet clinical meaningful endpoints in pivotal studies. Initial promise at Phase II of many proposed therapeutics was not translated into significant and meaningful clinical effect in subsequent pivotal Phase III clinical trials. Furthermore, there is a need for an SLE therapy that is efficacious across multiple organ domains.

Thus, there remains the need for safe and effective treatment of SLE that has proven clinical benefit, for example in a phase III double-blind, randomized, placebo controlled trial³³. SLE is a very heterogeneous disease and there further remains the need for a treatment of SLE manifestations that is effective across multiple organ systems, including musculoskeletal, mucocutaneous and immunologic domains.

The present invention solves one or more of the above-mentioned problems.

### 2 SUMMARY

The present invention relates to a method of treating or preventing mucocutaneous, musculoskeletal and/or renal disease in an systemic lupus erythematosus (SLE) patient in need thereof, the method comprising administering a therapeutically effective amount of a type I IFN receptor (IFNAR1) inhibitor to the patient, wherein the method treats mucocutaneous, musculoskeletal and/or renal disease in the patient.

The invention is supported inter alia by data presented for the first time herein including post hoc analysis of the phase 2 MUSE trial and the phase 3 TULIP-1 and TULIP-2 trials (NCT01438489, NCT02446912 and NCT02446899 respectively). The data show that, compared with placebo, treatment with a type I IFN receptor inhibitor in patients with moderate to severe SLE is associated with improvements across multiple organ systems, as measured by BILAG-2004 and SLEDAI-2K domain scores. In addition, more patients receiving the type I IFN receptor inhibitor compared with placebo had reductions in skin disease and swollen and tender joint counts. In addition, the type I IFN receptor inhibitor treated rash and arthritis in SLE patients. Together, these results provide evidence of the benefit of anifrolumab for reduction of disease activity across multiple organ domains in patients with active SLE. The type I IFN receptor inhibitor particular treats mucocutaneous, musculoskeletal and/or renal disease in the patient.

The invention also relates to the treatment of SLE patient in need thereof, the method comprising administering a therapeutically effective amount of a type I IFN receptor (IFNAR1) inhibitor to the patient, wherein the subject has low complement at baseline compared to a healthy subject, wherein the method reduces SLE disease activity in the subject. The invention is supported inter alia by data presented for the first time herein including post hoc analysis of the phase 3 TULIP-1 and TULIP-2 trials (NCT02446912 and NCT02446899 respectively). These data show that, anifrolumab response rates were higher in patients with baseline abnormal serologies vs those with normal serologies.

The invention also relates to the treatment of SLE patient in need thereof, the method comprising administering a therapeutically effective amount of a type I IFN receptor (IFNAR1) inhibitor to the patient, wherein them subject has treatment-refractory SLE and wherein the method reduces SLE disease activity in the subject. The invention is supported inter alia by data presented for the first time herein including post hoc analysis of the phase 3 TULIP-1 and TULIP-2 trials (NCT02446912 and NCT02446899 respectively). These data show that, there were consistently higher BICLA response rates with anifrolumab than with placebo, regardless of SLE standard therapy usage, including in patients with treatment-refractory SLE.

### 3 BRIEF DESCRIPTION OF THE DRAWINGS

FIG. 1: Distribution of IFN transcript scores

FIG. 2: MUSE follow-up

FIG. 2A: Patients were required to complete a 12-wk follow-up period and visits were conducted every 4 wks (±7 days) after the final study dose. FIG. 2B: IFNGS neutralisation—change in neutralization ratio of the 21-gene type I IFNGS³⁴ from start of the MUSE trial to the end of follow-up (week 60). From Wk 52 to Wk 60, IFNGS expression increased more rapidly in the anifrolumab 300-mg group vs the 1000-mg group.

FIG. 3: Efficacy in the MUSE trial

FIG. 3A: Disease activity measures at MUSE trial efficacy endpoint (week 52) and at end of follow-up (week 60). From Wk 52 to the end of the follow-up period (Wk 60), mean global SLEDAI-2K scores increased in patients coming off anifrolumab 300 mg and 1000 mg but not for the placebo group. A similar trend was observed in mean global BILAG-2004 scores in patients coming off anifrolumab 300 mg vs placebo. Mean CLASI scores increased slightly from Wk 52 to Wk 60 across the anifrolumab 300-mg, 1000-mg, and placebo groups. Disease activity, measured using MDGA score (patient reported outcomes), increased between Week 52 and Week 60 in both anifrolumab 300-mg and 1000-mg groups; there was no change in the placebo group. Active joint counts increased slightly from Week 52 to Week 60 across the anifrolumab 300-mg, anifrolumab 1000-mg, and placebo groups. FIG. 3B: Number of flares from MUSE trial efficacy endpoint (week 52) to end of follow-up (week 60). More patients ceasing treatment of anifrolumab 300 or 1000 mg had ≥1 BILAG flare from Week 52 through Week 60 versus placebo

FIG. 4: Efficacy in mucocutaneous and musculoskeletal organ domains

Change in Percentages of Patients With BILAG-2004 Scores A/B and C/D/E in the Mucocutaneous and Musculoskeletal Domains From MUSE Trial Efficacy Endpoint (Week 52) to End of Follow-up (Week 60). Mucocutaneous (left) was the most frequent organ system associated with worsening in patients ceasing anifrolumab, with shifts in the percentages of patients with BILAG C/D/E scores to BILAG A/B scores; similar trends were also observed in the musculoskeletal organ system. Worsening was most frequent in the mucocutaneous domain in patients coming off anifrolumab, with shifts in the percentages of patients with BILAG-2004 C/D/E to A/B scores

FIG. 5: Baseline patient demographics, disease characteristics and SLE medications

In the anifrolumab and placebo arms, the majority of patients had ≥1 BILAG A organ domain score or no A items and >2 B items.

FIG. 6: Baseline organ domain scores

Baseline BILAG-2004 (FIG. 6A) and SLEDAI-2K organ involvement (FIG. 6B), and BILAG-2004 organ domain (FIG. 6C) scores. Pb=Placebo; ANI=anifrolumab. The most commonly affected organ domains at baseline were mucocutaneous, musculoskeletal, and immunologic. Central nervous system (CNS)/neuropsychiatric and renal involvement were relatively uncommon at baseline for both BILAG-2004 and SLEDAI-2K because of the exclusion of patients with severe active lupus nephritis or severe active CNS manifestations. Baseline organ domain involvement assessed by BILAG-2004 and SLEDAI-2K was similar between treatment groups.

FIG. 7: BILAG-2004 responders at Week 52 by organ domain in TULIP-1 and TULIP-2

At Week 52, a greater number of patients treated with anifrolumab vs placebo had improvements in the BILAG-2004 mucocutaneous and musculoskeletal domain scores. Improvements were also observed in the majority of less frequently affected domains.

FIG. 8: Flares at Week 52 by Maintained OCS Dosage Reduction in Patients With Baseline OCS Dosage ≥10 mg/day in TULIP-1 and TULIP-2.

BILAG, British Isles Lupus Assessment Group; OCS, oral corticosteroid; SLEDAI, SLE Disease Activity Index. Maintained OCS dosage reduction was defined as OCS dosage of ≤7.5 mg/day achieved by Week 40 and maintained to Week 52. OCS are described as “Prednisone or equivalent.” OCS administered when necessary are not considered in the calculation of the daily dose. Flares were defined as ≥1 new BILAG-2004 A or ≥2 new BILAG-2004 B domain scores versus the prior visit. Randomization in TULIP-1 and TULIP-2 was stratified by OCS dosage (<10 versus ≥10 mg/day), SLEDAI-2K score (<10 versus ≥10), and type I interferon gene signature (high versus low).

FIG. 9: Efficacy of anifrolumab in rash and arthritis

FIG. 9A: Patients with SLEDAI-2K-defined resolution. Overall, more anifrolumab-treated patients versus placebo achieved SLEDAI-2K-defined complete resolution of rash. FIG. 9B: Patients with BIILAG-defined improvement in rash. The more sensitive measure, BILAG, which required an improvement of ≥1 grade, showed a benefit of anifrolumab over placebo for rash (difference 15.5%, nominal P<0.001); results were comparable in the IFNGS test-high subset. FIG. 9C: Patients with ≥50% improvement in mCLASI score from baseline to Week 52 (mCLASI >0 at baseline). Improvements of 250% from baseline to Week 52, defined by mCLASI, in patients with baseline mCLASI activity scores >0, were more frequent with anifrolumab versus placebo. FIG. 9D: Patients with SLEDAI-2K-defined resolution in arthritis. Overall, more anifrolumab-treated patients versus placebo achieved SLEDAI-2K-defined complete resolution in arthritis. FIG. 9E: Patients with BILAG-defined improvement in arthritis. Overall, more anifrolumab-treated patients versus placebo achieved BILAG-defined complete resolution in arthritis. FIG. 9F: Patients with ≥50% improvement in the number of swollen or tender joints from baseline to Week 52 (at baseline). Anifrolumab's efficacy was further confirmed by a ≥50% improvement in swollen and tender joint counts, in patients with ≥6 at baseline; the effect was comparable in IFNGS test-high and test-low patients. Data pooled from TULIP-1 and TULIP-2 trials. Resolution defined as: SLEDAI-2K rash component=0 (patients with SLEDAI-2K rash component=2 at baseline); SLEDAI-2K arthritis component=0 (patients with SLEDAI-2k arthritis component=4 at baseline); Improvement defined as: BILAG rash, mucocutaneous baseline score A change to B, C or D, or baseline score B change to C or D; BILAG arthritis, musculoskeletal baseline score A change to B, C, or D, or baseline B change to C or D. mCLASI defined as the activity portions of CLASI that describe skin erythema, scale/hypertrophy, and inflammation of the scalp. BILAG, British Isles Lupus Assessment Group; IFNGS, interferon gene signature; mCLASI, modified Cutaneous Lupus Erythematosus Disease Area and Severity Index; SLEDAI-2K, Systemic Lupus Erythematosus Disease Activity Index 2000.

FIG. 10: Baseline organ domain involvement assessed using BILAG-2004 and SLEDAI-2K

Baseline organ domain involvement assessed using BILAG-2004 (FIG. 10A) and SLEDAI-2K (FIG. 10B) was similar between treatment groups. BILAG-2004, British Isles Lupus Assessment Group-2004; SLEDAI-2K, Systemic Lupus Erythematosus Disease Activity Index 2000. BILAG-2004 scores range from level A (severe/active disease) to E (no current or previous disease). BILAG-2004 organ domain involvement was defined as an A or B score. SLEDAI-2K organ domain involvement was defined as any SLEDAI-2K organ system score. ^{a}Excluding fever.

FIG. 11: BILAG organ domain scores

BILAG organ domain scores were balanced across treatment groups. BILAG-2004 scores range from level A (severe/active disease) to E (no current or previous disease). BILAG-2004 organ domain involvement was defined as an A or B score. SLEDAI-2K organ domain involvement was defined as any SLEDAI-2K organ system score. ^{a}Excluding fever.

FIG. 12: Heat maps of individual patient BILAG-2004 organ domain scores over time, musculoskeletal and mucocutaneous

Patients with BILAG-2004 organ domain involvement at baseline, sorted by baseline score (A or B) and Week 52 score. Each row represents an individual patient and each column represents a BILAG-2004 organ domain score every 4 weeks from Week 0 (baseline) to Week 52. Colours indicate patient BILAG-2004 scores from dark grey (A, severe/active disease) to light grey (D, no current disease).

FIG. 13: Heat maps of individual patient BILAG-2004 organ domain scores over time, renal and cardiorespiratory

Patients with BILAG-2004 organ domain involvement at baseline, sorted by baseline score (A or B) and Week 52 score. Each row represents an individual patient and each column represents a BILAG-2004 organ domain score every 4 weeks from Week 0 (baseline) to Week 52. Colours indicate patient BILAG-2004 scores from dark grey (A, severe/active disease) to light grey (D, no current disease).

FIG. 14: Heat maps of individual patient BILAG-2004 organ domain scores over time, constitutional, hematologic, ophthalmic, neuropsychiatric and gastrointestinal

Patients with BILAG-2004 organ domain involvement at baseline, sorted by baseline score (A or B) and Week 52 score. Each row represents an individual patient and each column represents a BILAG-2004 organ domain score every 4 weeks from Week 0 (baseline) to Week 52. Colours indicate patient BILAG-2004 scores from dark grey (A, severe/active disease) to light grey (D, no current disease).

FIG. 15: BILAG-2004 responders at Week 52 by number of score shifts

A 1-score shift is a shift from an A score at baseline to a B score at Week 52 or a B score at baseline to a C score at Week 52; a 2-score shift is from A to C or B to D; a 3-score shift is from A to D. Improvement in BILAG-2004 organ domain scores was defined as a step down from an A or B score to a B, C, or D score among patients with an A or B score at baseline. BILAG responders are the patients with improvements from baseline at Week 52. **P<0.01; ***P<0.001 (based on Cochran-Mantel-Haenszel approach for the comparison of BILAG-2004 responder rates for anifrolumab vs placebo).

FIG. 16: BILAG-2004 organ domain responders overtime

Improvements favouring anifrolumab for the mucocutaneous and musculoskeletal BILAG-2004 domains were observed from Week 4 and Week 32, respectively. BILAG-2004, British Isles Lupus Assessment Group-2004. BILAG-2004 organ domain responder is defined as a reduction in baseline A or B score at Week 52. BILAG-2004 neuropsychologic, gastrointestinal, hematologic and ophthalmic domains are not plotted because there were too few patients in each treatment group. Points are estimates. Estimates are calculated using a stratified Cochran-Mantel-Haenszel approach, with stratification factors as listed in the Methods section. *P<0.05; **P<0.01; ***P<0.001 (based on Cochran-Mantel-Haenszel approach for the comparison of anifrolumab vs placebo).

FIG. 17: SLEDAI-2K organ domain responders over time

SLEDAI-2K organ domain responder is defined as a reduction in baseline SLEDAI-2K organ domain score. SLEDAI-2K central nervous system domain is not plotted because there were too few patients in each treatment group. Points are estimates. Estimates are calculated using a stratified Cochran-Mantel-Haenszel approach, with stratification factors as listed in the Methods section. *P<0.05; **P<0.01; ***P<0.001 (based on Cochran-Mantel-Haenszel approach for the comparison of anifrolumab vs placebo).

FIG. 18: Percentages of patients who achieved ≥50% reductions from baseline CLASI-A over time and baseline swollen joint count and tender joint count over time

CLASI response is defined as ≥50% reduction in CLASI-A from baseline for patients with baseline CLASI-A ≥10. Swollen and tender joint count responses are defined as ≥50% reduction in swollen or tender joint count respectively for patients with baseline counts of ≥6 or ≥8. Points are estimates. Estimates are calculated using a stratified Cochran-Mantel-Haenszel approach, with stratification factors as listed in the Methods section. *P<0.05; **P<0.01; ***P<0.001 (based on Cochran-Mantel-Haenszel approach for the comparison of anifrolumab vs placebo).

FIG. 19: BICLA response at week 52 by subgroup in the TULIP-2 and TULIP-1 trials

TI, TULIP I; TII, TULIP II; BICLA, BILAG-based Composite Lupus Assessment; C3, third component of complement; C4, fourth component of complement; CI, confidence interval; CMH, Cochran-Mantel-Haenszel; N, number of patients in treatment group; n, number of responders. The responder rates (percentages) and associated 95% CI are weighted and are calculated using a stratified CMH approach, with stratification factors (SLEDAI-2K score at screening [<10 points versus 10 points], Week 0 oral glucocorticoid dose [<10 mg/day prednisone or equivalent], and type I IFN gene signature test result at screening [high vs low]). Percentages are based upon all subjects in the full analysis set within subgroups. Baseline is defined as the last measurement prior to randomization and dose administration on Day 1.

FIG. 20: Forest plot of BICLA response according to baseline standard therapy in patients with SLE in TULIP-1 and TULIP-2

BICLA, BILAG-based Composite Lupus Assessment; CI, confidence interval; GC, glucocorticoid; IFNGS, interferon gene signature; n, number of responders; N, number of patients in th group; PGA, Physicians' Global Assessment. A BICLA response required: Reduction of all baseline BILAG-2004 A or 22 new BILAG-2004 B0; no increase in SLEDAI-2K score from baseline; no increase in ≥0.3 points in PGA score from baseline; no use of restricted medications beyond protocol-allowed thresholds, and no discontinuation of investigational product. The response rates, the differences in response rates, and associated 95% Cis were calculated using a stratified Cochran-Mantel-Haenszel method with stratification factors of SLEDAI-2K score at screening (<10 vs ≥10), baseline oral GC dosage (<10 vs ≥10 mg/day prednisone or equivalent), IFNGS status (high vs low), and study. ^{a}Prednisone or equivalent. ^{b}lmmunosuppressants were ≥1 or: azathioprine, methotrexate, mycophenolate, or mycophenolic acid.

FIG. 21. Delivery device

Anifrolumab is administered by an injection device [1] [9] such as a prefilled syringe (PFS) (FIG. 21A) or an autoinjector (AI) (FIG. 21B).

FIG. 22. Autoinjector

The autoinjector for administering anifrolumab of the functional variant thereof in exploded view (FIG. 22A), assembled (FIG. 22B) and filled with drug substance (FIG. 22C).

FIG. 23. Accessorized pre-filled syringe

The accessorized pre-filled syringe (APFS) for anifrolumab of the functional variant thereof. The primary tube is shown in assembled form (FIG. 23A) and in exploded view (FIG. 23B). The APFS with its additional components is shown in assembled form (FIG. 23C) and in exploded view FIG. 23D).

FIG. 24. Packaging for the delivery device

### 4 DETAILED DESCRIPTION

The invention relates to a method of treating or preventing mucocutaneous, musculoskeletal and/or renal disease in a systemic lupus erythematosus (SLE) patient in need thereof, the method comprising administering a therapeutically effective amount of a type I IFN receptor (IFNAR1) inhibitor to the patient, wherein the method treats mucocutaneous, musculoskeletal and/or renal disease in the patient. The method may treat mucocutaneous, musculoskeletal and renal disease in the patient. The method may, reduce the mucocutaneous, musculoskeletal and/or renal flare rate in the patient relative to pre-treatment mucocutaneous, musculoskeletal and/or renal flare rate respectively. The method may improves the patient's BILAG-2004 mucocutaneous, renal and/or musculoskeletal organ domain score. The method may improve the patient's SLEDAI-2K mucocutaneous and/or musculoskeletal organ domain score. The method may treat cardiorespiratory disease in the patient, optionally wherein the method improves the patient's BILAG-2004 cardiorespiratory organ domain score. The method may treat constitutional disease in the patient, optionally wherein the method improves the patient's BILAG-2004 constitutional organ domain score. The method may treat vascular, hematologic, renal and/or cardiorespiratory disease in the patient, optionally wherein the method improves the patient's SLEDAI-2K vascular, hematologic, renal and/or cardiorespiratory disease organ domain score.

The method may treat rash in the patient. There may be a ≥50% improvement in rash in the subject from pre-treatment levels of rash, optionally wherein the improvement is defined by Cutaneous Lupus Erythematosus Disease Area and Severity Index (CLASI). The method may resolve rash in the patient. The method may completely resolve SLEDAI-2K-defined rash in the patient.

The method may treat or prevent arthritis in the patient. The method may completely resolve arthritis in the patient, optionally wherein the method complete resolves SLEDAI-2K-defined arthritis in the patient.

The method may lead to a ≥50% improvement in swollen and tender joint count in the patient compared to the pre-treatment swollen and tender joint count in the patient. The patient may have ≥6 swollen and tender joint count pre-treatment.

The method may comprise treating or preventing renal disease in the patient, wherein the method treats or prevents renal disease in the patient. The patient may have a 24-hour UPCR >0.5 mg/mg pre-treatment, and wherein the method improves the subject's 24-hour UPCR to ≤0.5 mg/mg.

The invention also relates to a a method of treating SLE in a patient thereof, the method comprising administering a therapeutically effective amount of a type I IFN receptor (IFNAR1) inhibitor to the patient, wherein the patient has a baseline CLASI-A ≥10, wherein treatment reduces the patient's CLASI-A ≥50%. The treatment may reduce the patient's CLASI-A by at least week 12 of treatment. The method may lead to a reduction in the patient's CLASI-A that is maintained for at least 4, 8, 12, 16, 20, 24, 28, 32, 36 or 40 weeks.

The invention also relates to a method of treating a systemic lupus erythematosus (SLE) patient in need thereof, the method comprising administering a therapeutically effective amount of a type I IFN receptor (IFNAR1) inhibitor to the patient, wherein them subject has low complement at baseline compared to a healthy subject, wherein the method reduces SLE disease activity in the patient. Low complement may be defined as less than about 0.1 g/L C4 in the blood and/or less than about 0.9 g/L C3 in the blood.

The subject may have low C3 and/or C4 complement at baseline compared to a healthy subject. Low C3 may be defined as less than 0.9 g/L in the blood. Low C4 may be defined as less than 0.1 g/L in the blood. The subject may have a SLEDAI-2K score of ≥6, ≥1 A and/or a ≥2 B BILAG-2004 organ domain score, and/or a Physician's Global Assessment of ≥1.

The invention also relates to a method of treating a systemic lupus erythematosus (SLE) patient in need thereof, the method comprising administering a therapeutically effective amount of a type I IFN receptor (IFNAR1) inhibitor to the patient, wherein them subject has treatment-refractory SLE, and wherein the method reduces SLE disease activity in the subject. The subject may have previously received prior treatment with glucocorticoids, antimalarials and/or immunosuppressants. The subject may have a SLEDAI-2K score of ≥6, ≥1 A and/or a ≥2 B BILAG-2004 organ domain score, and/or a Physician's Global Assessment of 21. The subject may have received prior treatment with azathioprine, mizoribine, mycophenolate mofetil, mycophenolic acid, and/or methotrexate.

Reducing SLE disease activity in the subject may comprise a BILAG-Based Composite Lupus Assessment (BICLA) response.

The patient may have moderate to severe SLE.

The methods of the invention may have been demonstrated in a phase III clinical trial.

The type I IFN receptor inhibitor may be anifrolumab or a functional variant thereof. The method may comprise administering a fixed dose of anifrolumab. The method may comprise administering about 300 mg to about 1000 mg of anifrolumab. The method may comprise administering about 300 mg anifrolumab. The method may comprise administering anifrolumab or the functional variant thereof at a dose of 300-1000 mg every four weeks (Q4WW), Anifrolumab or the functional variant thereof may be administered intravenously. The method may comprise administering anifrolumab or the functional variant thereof to the patient at a dose of 120 mg every week, optionally wherein anifrolumab or the functional variant thereof is administered subcutaneously.

The method may comprise steroid sparing in the patient, wherein the dose of the steroid administered to the patient is tapered from a pre-sparing dose at baseline to a post-sparing dose. The post-sparing dose may be 57.5 mg/day prednisone or prednisone equivalent dose. The pre-sparing dose may be 10 mg/day or prednisone equivalent dose. The steroid may comprise a glucocorticoid. The steroid may comprise an oral glucocorticoid. The steroid may be hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof.

The steroid may comprise prednisone.

The patient may be a type I interferon stimulated gene signature (IFNGS)-test high patient pre-treatment. The method may comprise identifying the patient as IFNGS-test high patient before administration of the IFNAR1 inhibitor.

The invention also relates to a pharmaceutical composition for use in any of the methods of the invention.

The invention also relates to an injection device comprising the pharmaceutical composition of the invention. The injection device may be a pre-filled syringe (PFS). The injection device may be an accessorized pre-filed syringe (AFPS). The injection device may be an auto-injector.

The invention also relates to a kit comprising the injection device of the invention and instructions for use. The instructions for use may comprise instructions for subcutaneous administration of the pharmaceutical composition or unit dose to the patient. The instructions for use may specify that the injection device, unit dose and/or pharmaceutical composition are for use in the treatment of SLE. The kit may comprise packaging, wherein the packaging is adapted to hold the injection device and the instructions for use. The instructions for use may be attached to the injection device.

The instructions for use may specify that that administration of the pharmaceutical composition to the patient treats mucocutaneous, musculoskeletal and/or renal disease in the patient. The instructions for use may specify that the pharmaceutical composition treats mucocutaneous, musculoskeletal and renal disease in the patient. The instructions for use may specify that the pharmaceutical composition reduces the mucocutaneous, musculoskeletal and/or renal flare rate in the patient relative to pre-treatment mucocutaneous, musculoskeletal and/or renal flare rate respectively.

The instructions for use may specify that administration of the pharmaceutical composition to the patient improves the patient's BILAG-2004 mucocutaneous, renal and/or musculoskeletal organ domain score.

The instructions for use may specify that administration of the pharmaceutical composition to the patient improves the patient's SLEDAI-2K mucocutaneous and/or musculoskeletal organ domain score.

The instructions for use may specify that that administration of the pharmaceutical composition to the patient treats cardiorespiratory disease in the patient. The instructions for use may specify that the pharmaceutical composition improves the patient's BILAG-2004 cardiorespiratory organ domain score.

The instructions for use may specify that that administration of the pharmaceutical composition to the patient treats constitutional disease in the patient. The instructions for use may specify that the pharmaceutical composition improves the patient's BILAG-2004 constitutional organ domain score.

The instructions for use may specify that administration of the pharmaceutical composition treats vascular, hematologic, renal and/or cardiorespiratory disease in the patient. The instructions for use may specify that the pharmaceutical composition improves the patient's SLEDAI-2K vascular, hematologic, renal and/or cardiorespiratory disease organ domain score.

The instructions for use may specify that administration of the pharmaceutical composition treats rash in the patient, optionally there is a ≥50% improvement in rash in the subject from pre-treatment levels of rash, optionally wherein the improvement is defined by Cutaneous Lupus Erythematosus Disease Area and Severity Index (CLASI).

The instructions for use may specify that administration of the pharmaceutical composition to the patient resolves rash in the patient, and optionally wherein that administration with the pharmaceutical composition completely resolves SLEDAI-2K-defined rash in the patient.

The instructions for use may specify that that administration of the pharmaceutical composition to the patient treats or prevent arthritis in the patient.

The instructions for use may specify that that administration of the pharmaceutical composition to the patient completely resolves arthritis in the patient, optionally wherein the pharmaceutical composition completely resolves SLEDAI-2K-defined arthritis in the patient.

The instructions for use may specify that administration of the pharmaceutical composition leads to a ≥50% improvement in swollen and tender joint count in the patient compared to the pre-treatment swollen and tender joint count in the patient, optionally wherein the patient had ≥6 swollen and tender joint count pre-treatment.

The instructions for use may specify that that administration of the pharmaceutical composition to the patient treats or prevents renal disease in the patient.

The instructions for use may specify that that administration of the pharmaceutical composition to the patient reduces the patient's CLASI-A ≥50%, optionally wherein the patient has a baseline CLASI-A ≥10. The instructions for use may specify that that administration of the pharmaceutical composition to the patient reduces the patient's CLASI-A by at least week 12 of treatment, optionally wherein the reduction in the patient's CLASI-A is maintained for at least 4, 8, 12, 16, 20, 24, 28, 32, 36 or 40 weeks.

The instructions for use may specify that the patient has low complement at baseline compared to a healthy subject, and that administration of the pharmaceutical composition to the patient reduces SLE disease activity in the patient.

The instructions for use may specify that the patient has low C3 and/or C4 complement at baseline compared to a healthy subject.

The instructions for use may specify that the patient has treatment-refractory SLE, and and that administration of the pharmaceutical composition to the patient reduces SLE disease activity in the patient.

The instructions for use may specify that the patient has previously received prior treatment with glucocorticoids, antimalarials and/or immunosuppressants.

The instructions for use may specify that pre-treatment the patient has a SLEDAI-2K score of ≥6, ≥1 A and/or a ≥2 B BILAG-2004 organ domain score, and/or a Physician's Global Assessment of ≥1.

The instructions for use may specify that the patient has received prior treatment with azathioprine, mizoribine, mycophenolate mofetil, mycophenolic acid, and/or methotrexate.

### Definitions

### 5.1 Type I IFN Receptor Inhibitor

A “type I interferon receptor inhibitor” refers to a molecule that is antagonistic for the receptor of type I interferon ligands such as interferon-α and interferon-β. Such inhibitors, subsequent to administration to a patient, preferably provide a reduction in the expression of at least 1 (preferably at least 4) pharmacodynamic (PD) markergenes selected from the group consisting of IF16, RSAD2, IF144, IF144L, IF127, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IF127, IF144, IF144L, and RSAD2. The “type I interferon receptor” is preferably interferon-α/β receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist.

Additionally or alternatively, the type I interferon receptor inhibitor may be a small molecule inhibitor of a type I interferon receptor (e.g. for pharmacological inhibition of type I interferon receptor activity).

The type I interferon receptor inhibitor may be an antibody or antigen-binding fragment thereof that inhibits type I IFN activity. A particularly preferred type I interferon receptor inhibitor is the antibody anifrolumab or a functional variant thereof. Anifrolumab is a monoclonal antibody targeting IFNAR1 (the receptor for α, β, and ω interferons). Disclosure related to anifrolumab can be found in U.S. Pat. Nos. 7,662,381 and 9,988,459, which are incorporated herein by reference.

Anifrolumab is a monoclonal antibody which binds to IFNAR with high affinity and specificity. The antibody is an IFNAR-blocking (antagonistic) antibody, and blocks the activity of the receptor's ligands, namely type I interferons such as interferon-α and interferon-β. Anifrolumab thus provides for downregulation of IFNAR signalling, and thus suppression of IFN-inducible genes.

TABLE 5-1
Anifrolumab sequences

| | |
|---|---|
| Anifrolumab VH (SEQ ID NO: 1) | EVQLVQSGAEVKKPGESLKISCKGSGYIFTNYWIAWVRQMPGKGLESMG |
| | IIYPGDSDIRYSPSFQGQVTISADKSITTAYLQWSSLKASDTAMYYCARHDI |
| | EGFDYWGRGTLVTVSS |
| Anifrolumab VK (SEQ ID NO: 2) | EIVLTQSPGTLSLSPGERATLSCRASQSVSSSFFAWYQQKPGQAPRLLIY |
| | GASSRATGIPDRLSGSGSGTDFTLTITRLEPEDFAVYYCQQYDSSAITFG |
| | QGTRLEIK |
| HCDR1 (SEQ ID NO: 3) | NYWIA |
| HCDR2 (SEQ ID NO: 4) | IIYPGDSDIRYSPSFQG |
| HCDR3 (SEQ ID NO: 5) | HDIEGFDY |
| LCDR1 (SEQ ID NO: 6) | RASQSVSSSFFA |
| LCDR2 (SEQ ID NO: 7) | GASSRAT |
| LCDR3 (SEQ ID NO: 8) | QQYDSSAIT |
| Light chain constant region | RTVAAPSVFIFPPSDEQLKSGTASVVCLLNNFYPREAKVQWKVDNALQS |
| (SEQ ID NO: 9) | GNSQESVTEQDSKDSTYSLSSTLTLSKADYEKHKVYACEVTHQGLSSPV |
| | TKSFNRGEC |
| Heavy chain constant region | ASTKGPSVFPLAPSSKSTSGGTAALGCLVKDYFPEPVTVSWNSGALTSG |
| (SEQ ID NO: 10) | VHTFPAVLQSSGLYSLSSVVTVPSSSLGTQTYICNVNHKPSNTKVDKRVE |
| | PKSCDKTHTCPPCPAPEFEGGPSVFLFPPKPKDTLMISRTPEVTCVVVDV |
| | SHEDPEVKFNWYVDGVEVHNAKTKPREEQYNSTYRVVSVLTVLHQDWL |
| | NGKEYKCKVSNKALPASIEKTISKAKGQPREPQVYTLPPSREEMTKNQVS |
| | LTCLVKGFYPSDIAVEWESNGQPENNYKTTPPVLDSDGSFFLYSKLTVDK |
| | SRWQQGNVFSCSVMHEALHNHYTQKSLSLSPGK |
| Heavy chain | EVQLVQSGAEVKKPGESLKISCKGSGYIFTNYWIAWVRQMPGKGLESMG |
| (SEQ ID NO: 11) | IIYPGDSDIRYSPSFQGQVTISADKSITTAYLQWSSLKASD TAMYYCARHD |
| | IEGFDYWGRGTLVTVSSASTKGPSVFPLAPSSKSTSGGTAALGCLVKDYF |
| | PEPVTVSWNSGALTSGVHTFPAVLQSSGLYSLSSVVTVPS SSLGTQTYIC |
| | NVNHKPSNTKVDKRVEPKSCDKTHTCPPCPAPEFEGGPSVFLFPPKPKD |
| | TLMISRTPEVTCVVVDVSHEDPEVKFNWYVDGVEVHNAKTK |
| | PREEQYNSTYRVVSVLTVLHQDWLNGKEYK CKVSNKALPA |
| | SIEKTISKAK |
| | GQPREPQVYTLPPSREEMTKNQVSLTCLVKGFYPSDIAVEWESNGQPEN |
| | NYKTTPPVLDSDGSFFLYSKLTVDKSRWQQGNVFSCSVMHEALHNHYT |
| | QKS LSLSPGK |
| Light chain | EIVLTQSPGTLSLSPGERATLSCRASQSVS SSFFAWYQQK |
| (SEQ ID NO: 12) | PGQAPRLLIY GASSRATGIPDRLSGSGSGT DFTLTITRLE PEDFAVYYCQ |
| | QYDSSAITFG QGTRLEIKRTVAAPSVFIFPPSDEQLKSGT ASVVCLLNNF |
| | YPREAKVQWK |
| | VDNALQSGNSQESVTEQDSKDSTYSLSSTLTLSKADYEKHKVYACEVTH |
| | QGLSSPVTKSFNRGEC |

Thus, “anifrolumab” is an antibody comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). In more detail, anifrolumab as referred to herein is an antibody comprising a VH of SEQ ID NO: 1 and a VL of SEQ ID NO: 2 (or functional variant thereof).

The constant region of anifrolumab has been modified such that anifrolumab exhibits reduced affinity for at least one Fc ligand compared to an unmodified antibody. Anifrolumab is a modified IgG class monoclonal antibody specific for IFNAR1 comprising in the Fc region an amino acid substitution of L234F, as numbered by the EU index as set forth in Kabat (1991, NIH Publication 91-3242, National Technical Information Service, Springfield, Va.). Anifrolumab is a modified IgG class monoclonal antibody specific for IFNAR1 comprising in the Fc region an amino acid substitution of L234F, L235E and/or P331S, as numbered by the EU index as set forth in Kabat (1991, NIH Publication 91-3242, National Technical Information Service, Springfield, Va.). Anifrolumab is an antibody comprising a light chain constant region of SEQ ID NO: 9. Anifrolumab is an antibody comprising a heavy chain constant region of SEQ ID NO: 10. Anifrolumab is an antibody comprising a light chain constant region of SEQ ID NO: 9 and a heavy chain constant region of SEQ ID NO: 10. Anifrolumab is an antibody comprising a heavy chain of SEQ ID NO: 11. Anifrolumab is an antibody comprising a light chain of SEQ ID NO: 12. Anifrolumab is an antibody comprising a heavy chain of SEQ ID NO: 11 and a light chain of SEQ ID NO: 12.

The present invention encompasses the antibodies defined herein having the recited CDR sequences or variable heavy and variable light chain sequences (reference (anifrolumab) antibodies), as well as functional variants thereof. A “functional variant” binds to the same target antigen as the reference (anifrolumab) antibody. The functional variants may have a different affinity for the target antigen when compared to the reference antibody, but substantially the same affinity is preferred. Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

Functional variants of a reference (anifrolumab) antibody may show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, preferably at most 1 amino acid differences when compared to a corresponding reference CDR sequence, and when combined with the remaining 5 CDRs (or variants thereof) enables the variant antibody to bind to the same target antigen as the reference (anifrolumab) antibody, and preferably to exhibit the same affinity for the target antigen as the reference (anifrolumab) antibody.

Without wishing to be bound by theory, since anifrolumab targets (e.g. blocks or antagonizes) IFNAR, it is believed that anifrolumab treats a disease (such as lupus nephritis) by blocking signalling initiated by type I interferons (IFNs). Type I IFNs are known to be important drivers of inflammation (e.g. by coordinating the type I interferon response), and thus play a pivotal role in the immune system. However, dysregulation of type I IFN-signalling can lead to aberrant (e.g. aberrantly high) levels of inflammation, and autoimmunity. Such dysregulation of type I IFN interferons has been reported in numerous autoimmune diseases.

A variant of the reference (anifrolumab) antibody may comprise: a heavy chain CDR1 having at most 2 amino acid differences when compared to SEQ ID NO: 3; a heavy chain CDR2 having at most 2 amino acid differences when compared to SEQ ID NO: 4; a heavy chain CDR3 having at most 2 amino acid differences when compared to SEQ ID NO: 5; a light chain CDR1 having at most 2 amino acid differences when compared to SEQ ID NO: 6; a light chain CDR2 having at most 2 amino acid differences when compared to SEQ ID NO: 7; and a light chain CDR3 having at most 2 amino acid differences when compared to SEQ ID NO: 8; wherein the variant antibody binds to the target of anifrolumab (e.g. IFNAR) and preferably with the same affinity.

A variant of the reference (anifrolumab) antibody may comprise: a heavy chain CDR1 having at most 1 amino acid difference when compared to SEQ ID NO: 3; a heavy chain CDR2 having at most 1 amino acid difference when compared to SEQ ID NO: 4; a heavy chain CDR3 having at most 1 amino acid difference when compared to SEQ ID NO: 5; a light chain CDR1 having at most 1 amino acid differences when compared to SEQ ID NO: 6; a light chain CDR2 having at most 1 amino acid difference when compared to SEQ ID NO: 7; and a light chain CDR3 having at most 1 amino acid difference when compared to SEQ ID NO: 8; wherein the variant antibody binds to the target of anifrolumab (e.g. IFNAR) optionally with the same affinity.

A variant antibody may have at most 5, 4 or 3 amino acid differences total in the CDRs thereof when compared to a corresponding reference (anifrolumab) antibody, with the proviso that there is at most 2 (optionally at most 1) amino acid differences per CDR. A variant antibody may have at most 2 (optionally at most 1) amino acid differences total in the CDRs thereof when compared to a corresponding reference (anifrolumab) antibody, with the proviso that there is at most 2 amino acid differences per CDR. A variant antibody may have at most 2 (optionally at most 1) amino acid differences total in the CDRs thereof when compared to a corresponding reference (anifrolumab) antibody, with the proviso that there is at most 1 amino acid difference per CDR.

The amino acid difference may be an amino acid substitution, insertion or deletion. The amino acid difference may be a conservative amino acid substitution as described herein.

A variant antibody may have at most 5, 4 or 3 amino acid differences total in the framework regions thereof when compared to a corresponding reference (anifrolumab) antibody, with the proviso that there is at most 2 (optionally at most 1) amino acid differences per framework region. Optionally a variant antibody has at most 2 (optionally at most 1) amino acid differences total in the framework regions thereof when compared to a corresponding reference (anifrolumab) antibody, with the proviso that there is at most 2 amino acid differences per framework region. Optionally a variant antibody has at most 2 (optionally at most 1) amino acid differences total in the framework regions thereof when compared to a corresponding reference (anifrolumab) antibody, with the proviso that there is at most 1 amino acid difference per framework region.

Thus, a variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains. A variant antibody may comprise a variable heavy chain and a variable light chain as described herein, wherein: the heavy chain has at most 7 amino acid differences (at most 1 amino acid difference in each CDR and at most 1 amino acid difference in each framework region) when compared to a heavy chain sequence herein; and the light chain has at most 7 amino acid differences (at most 1 amino acid difference in each CDR and at most 1 amino acid difference in each framework region) when compared to a light chain sequence herein; wherein the variant antibody binds to the same target antigen as the reference (anifrolumab) antibody (e.g. IFNAR) and preferably with the same affinity.

The term “anifrolumab” preferably encompasses an antigen binding fragment thereof. The term “antigen-binding fragment”, refers to one or more fragments of anifrolumab that retain(s) the ability to specifically bind to the antigen for anifrolumab (IFNAR). Examples of antigen-binding fragments include the following: Fab fragment, F(ab′)2 fragment, Fd fragment, Fv fragment, dAb fragment, as well as a scFv.

Thus, in one embodiment the type I interferon receptor inhibitor is anifrolumab or a functional variant thereof.

### 5.2 End Points

### 5.2.1 BILAG-2004 (British Isles Lupus Assessment Group-2004)

The BILAG-2004 is a translational index with 9 organ systems (General, Mucocutaneous, Neuropsychiatric, Musculoskeletal, Cardiorespiratory, Gastrointestinal, Ophthalmic, Renal and Haematology) that is able to capture changing severity of clinical manifestations. It has ordinal scales by design and does not have a global score; rather it records disease activity across the different organ systems at a glance by comparing the immediate past 4 weeks to the 4 weeks preceding them. It is based on the principle of physicians' intention to treat and categorises disease activity into 5 different levels from A to E:
Grade A represents very active disease requiring immunosuppressive drugs and/or a prednisone dose of ≥20 mg/day or equivalent
Grade B represents moderate disease activity requiring a lower dose of corticosteroids, topical steroids, topical immunosuppressives, antimalarials, or NSAIDs
Grade C indicates mild stable disease
Grade D implies no disease activity but the system has previously been affected
Grade E indicates no current or previous disease activity

Although the BILAG-2004 was developed based on the principle of intention to treat, the treatment has no bearing on the scoring index. Only the presence of active manifestations influences the scoring.

### 5.2.2 BICLA (BILAG-Based Composite Lupus Assessment)

BICLA is a composite index that was originally derived by expert consensus of disease activity indices. BICLA response is defined as (1) at least one gradation of improvement in baseline BILAG scores in all body systems with moderate or severe disease activity at entry (e.g., all A (severe disease) scores falling to B (moderate), C (mild), or D (no activity) and all B scores falling to C or D); (2) no new BILAG A or more than one new BILAG B scores; (3) no worsening of total SLEDAI score from baseline; (4) no significant deterioration (510%) in physicians global assessment; and (5) no treatment failure (initiation of non-protocol treatment).

Particularly, a subject is a BICLA responder if the following criteria are met:
a) Reduction of all baseline BILAG-2004 A to B/C/D and baseline BILAG-2004 B to C/D, and no BILAG-2004 worsening in other organ systems, as defined by 1 new BILAG-2004 A or more than 1 new BILAG-2004 B item;
b) No worsening from baseline in SLEDAI-2K as defined as an increase from baseline of ≥0 points in SLEDAI-2K;
c) No worsening from baseline in the subjects' lupus disease activity defined by an increase ≥0.30 points on a 3-point PGA VAS;
d) No discontinuation of investigational product or use of restricted medications beyond the protocol-allowed threshold before assessment

### 5.2.3 CLASI (Cutaneous Lupus Erythematosus Disease Area and Severity Index Inflammatory Disease Activity)

The Cutaneous Lupus Erythematosus Disease Area and Severity Index (CLASI) was developed in 2005 as a means of specifically tracking cutaneous activity and damage in patients with CLE³⁵. The CLASI is a simple, single-page tool that separately quantifies skin disease activity and damage in each part of the body³⁶. The CLASI features a skin activity summary score (CLASI-A) and damage summary score (CLASI-D). This index has a high inter-rater and intra-rater reliability and is responsive to change when used in adults with SLE. CLASI activity score correlates with the severity of disease: mild, moderate, and severe disease corresponded with CLASI activity score ranges of 0-9 (sensitivity 93%, specificity 78%), 10-20, and 21-70 (sensitivity 80%, specificity 95%), respectively (Table 5-2).

TABLE 5-2
Disease severity based on the CLASI activity score

| | CLASI activity score range |
|---|---|
| Mild | 0-9 |
| Moderate | 10-20 |
| Severe | 21-70 |

The Cutaneous Lupus Erythematosus Disease Area and Severity Index (CLASI) quantifies disease activity and damage in cutaneous lupus erythematosus. It can distinguish between different response levels of treatment, e.g., it is able to detect a specific percentage reduction in activity score from baseline, or can be reported by a mean/median score. Particularly, the CLASI is a validated index used for assessing the cutaneous lesions of lupus and consists of 2 separate scores: the first summarizes the inflammatory activity of the disease; the second is a measure of the damage done by the disease. The activity score takes into account erythema, scale/hypertrophy, mucous membrane lesions, recent hair loss, and nonscarring alopecia. The damage score represents dyspigmentation, scarring/atrophy/panniculitis, and scarring of the scalp. Subjects are asked if their dyspigmentation lasted 12 months or longer, in which case the dyspigmentation score is doubled. Each of the above parameters is measured in 13 different anatomical locations, included specifically because they are most often involved in cutaneous lupus erythematosus (CLE). The most severe lesion in each area is measured.

Modified CLASI (mCLASI) is defined as the activity portions of CLASI that describe skin erythema, scale/hypertrophy, and inflammation of the scalp. Activity of oral ulcers and alopecia without scalp inflammation are excluded from the mCLASI analysis, as are all measures of damage. Clinically meaningful improvement in rash, as measured using mCLASI, is defined by ≥50% decrease in baseline activity score.

### 5.2.4 Joint Count

The swollen and tender joint count is based on left and right shoulder, elbow, wrist, metacarpophalangeal (MCP) 1, MCP2, MCP3, MCP4, MCP5, proximal interphalangeal (PIP) 1, PIP2, PIP3, PIP4, PIP5 joints of the upper extremities and left and right knee of the lower extremities. Active joint for the joint count assessment is herein defined as a joint with tenderness and swelling only. Each of 28 joints will be then be evaluated separately for tenderness (by palpating the joint) and swelling.

### 5.2.5 Proteinuria

The urine protein/creatinine ratio (UPCR) provides a readout of the amount of blood protein that is passed into the urine. UPCR may be measured in a urine sample collected over a 24-hour period (24-hour UPCR). UPCR may be a spot UPCR, which provides the protein/creatinine ratio measured in a randomly collected urine sample to estimate 24-hour protein excretion.

### 5.2.6 SRI (Systemic Lupus Erythematosus Responder Index of ≥4)

A subject achieves SRI(4) if all of the following criteria are met:
Reduction from baseline of ≥4 points in the SLEDAI-2K;
No new organ system affected as defined by 1 or more BILAG-2004 A or 2 or more
BILAG-2004 B items compared to baseline using BILAG-2004;
No worsening from baseline in the subjects' lupus disease activity defined by an increase ≥0.30 points on a 3-point PGA VAS.

SRI(X) (X=5, 6, 7, or 8) is defined by the proportion of subjects who meet the following criteria:
Reduction from baseline of X points in the SLEDAI-2K;
No new organ systems affected as defined by 1 or more BILAG-2004 A or 2 or
more BILAG-2004 B items compared to baseline using BILAG-2004;
No worsening from baseline in the subjects' lupus disease activity defined by an
increase ≥0.30 points on a 3-point PGA VAS

### 5.2.7 SLEDAI-2K (Systemic Lupus Erythematosus Disease Activity Index 2000)

The SLEDAI-2K disease activity index consists of a list of organ manifestations, each with a definition. A certified Investigator or designated physician will complete the SLEDAI-2K assessment and decide whether each manifestation is “present” or “absent” in the last 4 weeks. The assessment also includes the collection of blood and urine for assessment of the laboratory categories of the SLEDAI-2K.

The SLEDAI-2K assessment consists of 24 lupus-related items. It is a weighted instrument, in which descriptors are multiplied by a particular organ's “weight”. For example, renal descriptors are multiplied by 4 and central nervous descriptors by 8 and these weighted organ manifestations are totaled into the final score. The SLEDAI-2K score range is 0 to 105 points with 0 indicating inactive disease. The SLEDAI-2K scores are valid, reliable, and sensitive clinical assessments of lupus disease activity. The SLEDAI-2K calculated using a timeframe of 30 days prior to a visit for clinical and laboratory values has been shown to be similar to the SLEDAI-2K with a 10-day window³⁷.

### 5.2.8 Patient Reported Outcomes

Physician Global Assessment (PGA and MDGA) of Disease Activity refers to an assessment wherein a physician evaluates the status of a subject's psoriatic arthritis (PsA) by means of a visual analog scale (VAS). The subject is assessed according to how their current arthritis is. The VAS is anchored with verbal descriptors of “very good” to “very poor.”

### 5.3 Clinical Trials

### 5.3.1 Phase 2/Phase II/Pivotal Studies

Phase II studies gather preliminary data on effectiveness. In Phase 2 studies, researchers administer the drug to a group of patients with the disease or condition for which the drug is being developed. Typically involving a few hundred patients, these studies aren't large enough to show whether the drug will be beneficial. Instead, Phase 2 studies provide researchers with additional safety data. Researchers use these data to refine research questions, develop research methods, and design new Phase 3 research protocols.

### 5.3.2 Phase 3/Phase III/Pivotal Studies or Trials

Researchers design Phase 3 studies to demonstrate whether or not a product offers a treatment benefit to a specific population. Sometimes known as pivotal studies, these studies involve 300 to 3,000 participants. Phase 3 studies provide most of the safety data. In previous studies, it is possible that less common side effects might have gone undetected. Because these studies are larger and longer in duration, the results are more likely to show long-term or rare side effects. Regulatory bodies such as the EMA and FDA usually require a phase III clinical trial demonstrating that the product is safe and at least as effective (if not better) than available medications, before approving a new medication. Phase III clinical trials usually fail, even if they follow a successful a phase II clinical trial.

### 5.4 Delivery Device

The type I IFN inhibitor may be administered subcutaneously using an accessorized pre-filled syringe (APFS), an autoinjector (AI), or a combination thereof. Such devices have been found to be well-tolerated and reliable for administering subcutaneous doses of an antibody and provide further options for optimizing patient care. Indeed, such devices may reduce the burden of frequent clinic visits for patients. An example of a suitable APFS device is described in Ferguson et. al.³⁸, which is incorporated herein by reference in its entirety. The delivery device may be single use, disposable system that is designed to enable manual, SC administration of the dose.

### 5.5 Steroids

Steroids, particularly oral corticosteroids (OCS, glucocorticoids) include prednisone, cortisone, hydrocortisone, methylprednisolone, prednisolone and triamcinolone. Examples of equivalent doses of oral prednisone are shown in Table 5-3.

TABLE 5-3
Examples of equivalent doses of oral prednisone
Oral Prednisone and Equivalents

| Oral | Equivalent Dose | | | | | | | | | |
|---|---|---|---|---|---|---|---|---|---|---|
| Prednisone | 7.5 mg | | 10 mg | | 20 mg | | 30 mg | | 40 mg | |
| Cortisone | 37.5 | mg | 50 | mg | 100 | mg | 150 | mg | 200 | mg |
| Hydrocortisone | 30 | mg | 40 | mg | 80 | mg | 120 | mg | 160 | mg |
| Methylpred- | 6 | mg | 8 | mg | 16 | mg | 24 | mg | 32 | mg |
| nisolone | | | | | | | | | | |
| Prednisolone | 7.5 | mg | 10 | mg | 20 | mg | 30 | mg | 40 | mg |
| Triamcinolone | 6 | mg | 8 | mg | 16 | mg | 24 | mg | 32 | mg |

### 5.6 Type I IFN Gene Signature (IFNGS)

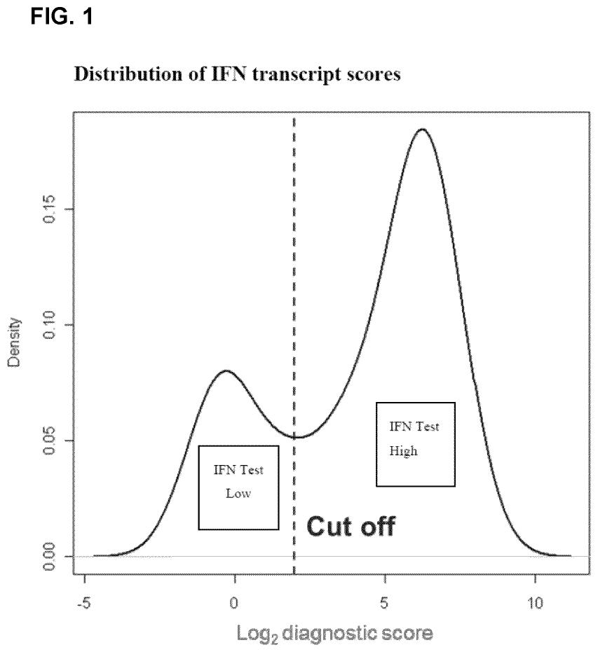

Type I IFN is considered to play a central role SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of type I IFN remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation (FIG. 1). The type I IFN test is described in WO201 1028933 A1, which is incorporated herein by reference in its entirety. The type I IFN gene signature may be used to identify a subject has a type I IFN gene signature (IFNGS)-test high patient or an IFNGS-test low patient. The IFNGS test measures expression of the genes IF127, IFI44, IFI44L, and RSAD2 compared with 3 reference genes; 18S, ACTB and GAPDH in the whole blood of the subject. The result of the test is a score that is compared with a pre-established cut-off that classifies patients into 2 groups with low or high levels of IFN inducible gene expression (FIG. 1).

The expression of the genes may be measured by RT-PCR. Suitable primers and probes for detection of the genes may be found in WO201 1028933. A suitable kit for measuring gene expression for the IFNGS test is the QIAGEN Therascreen® IFIGx RGQ RT-PCR kit (IFIGx kit), as described in Brohawn et al.³⁹, which is incorporated herein by reference in its entirety.

### 5.7 Formulations

Stable formulations suitable for administration to subjects and comprising anifrolumab are described in detail in U.S. patent Ser. No. 10/125,195 B1, which is incorporated herein in its in entirety.

The Examples that follow are illustrative of specific embodiments of the disclosure, and various uses thereof. They are set forth for explanatory purposes only and should not be construed as limiting the scope of the disclosure in any way.

### 6 Example 1: MUSE, ClinicalTrial.gov Identifier: NCT01438489

MUSE was a Phase 2, multinational, multicentre, randomized, double-blind, placebo controlled, parallel-group study to evaluate the efficacy and safety of 2 intravenous (IV) treatment regimens in adult participants with chronic, moderately-to-severely active SLE with an inadequate response to standard of care (SOC) SLE. The investigational product (anifrolumab or placebo) was administered as a fixed dose every 4 weeks (28 days) for a total of 13 doses.

MUSE is described in further detail in Furie et al. 201729, which is incorporated herein by reference in its entirety.

### 7 Example 2: TULIP I and II, ClinicalTrial.Gov Identifiers: NCT02446912 and NCT02446899

TULIP I and TULIP II were Phase 3, multicentre, multinational, randomised, double-blind, placebo-controlled studies to evaluate the efficacy and safety of an intravenous (IV) treatment regimen of two doses of anifrolumab versus placebo in subjects with moderately to severely active, autoantibody-positive systemic lupus erythematosus (SLE) while receiving standard of care (SOC) treatment.

### 7.1.1 Restricted Medications

If a subject received 1 of the following, the subject was considered a non-responder. Sulfasalazine; Danazol; Dapsone; Azathioprine >200 mg/day or at a daily dose greater than that at Week 0 (Day 1); Mycophenolate mofetil >2.0 g/day or mycophenolic acid >1.44 g/day or at a daily; dose greater than that at Week 0 (Day 1); Oral, SC, or intramuscular methotrexate >25 mg/week or at a daily dose greater than that at Week 0 (Day 1); Mizoribine >150 mg/day or at a daily dose greater than that at Week 0 (Day 1); Any change in route of administration of oral, SC, or intramuscular methotrexate; Intravenous corticosteroids >40 mg/day but:1 gm/day methylprednisolone or equivalent; Intramuscular corticosteroids >80 mg/day methylprednisolone or equivalent; Subcutaneous or intramuscular corticosteroid precursors; Treatment with OCS >40 mg/day prednisone or equivalent; Treatment with OCS above Day 1 dose for a dosing period >14 days; Corticosteroids with a long biologic half-life (eg, dexamethasone, betamethasone); Other immunosuppressants including but not limited to calcineurin inhibitors (eg, cyclosporine, tacrolimus [including topical]) or leflunomide. Cyclosporine eye drops were acceptable for use in the study.

TULIP I is described in further detail in Furie et al. 2019³⁰, which is incorporated herein by reference in its entirety. The results of TULIP II are presented in Morand et al. 2020²⁸, herein incorporated by reference in its entirety.

### 8 Example 3: Disease Activity in Patients with SLE Coming Off Anifrolumab During the 12-Week Follow-Up Period of the Phase 2b MUSE Trial

### 8.1 Introduction

In the MUSE trial (see Section 6), anifrolumab treatment reduced disease activity vs placebo across multiple endpoints in patients with moderately to severely active SLE. The inventors assessed for the first time the safety and efficacy in patients coming off anifrolumab during the 12-week (wk) follow-up period in MUSE.

### 8.2 Methods

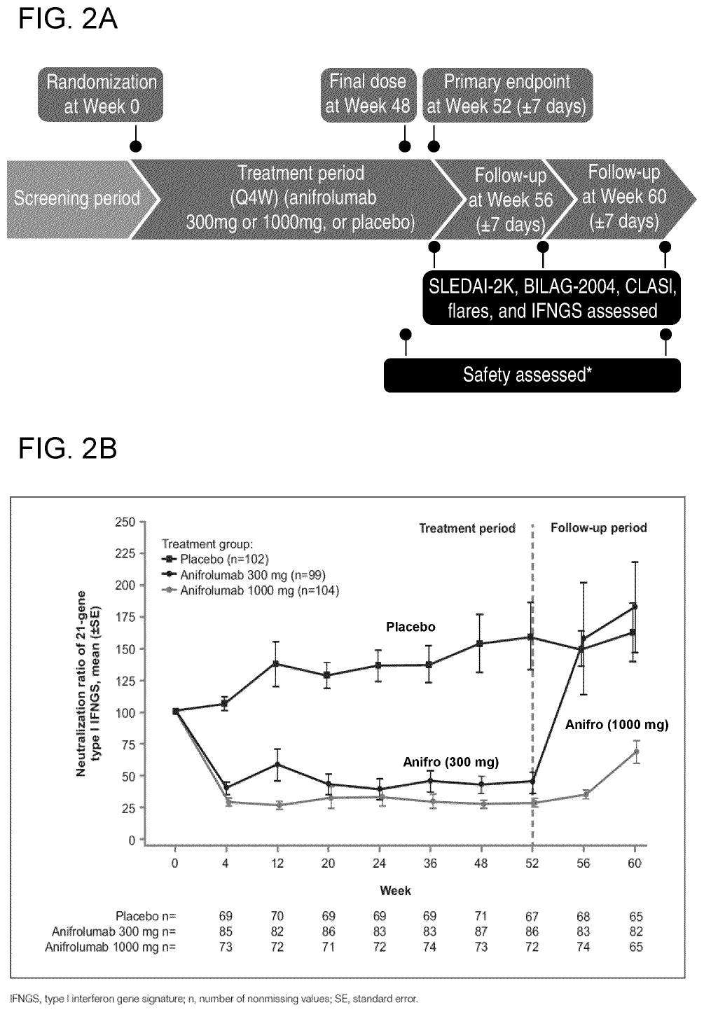

Patients were randomized 1:1:1 to receive placebo or anifrolumab 300 or 1000 mg every 4 wks; final study dose was Wk 48 and key efficacy endpoints were assessed at Wk 52. Patients were required to complete a 12-wk follow-up period and visits were conducted every 4 wks (±7 days) after the final study dose (FIG. 2A). Disease activity was measured using SLEDAI-2K and BILAG-2004. Flares were defined as either ≥1 new BILAG-2004 A or ≥2 new BILAG-2004 B items. Adverse events (AEs) and changes in the 21-gene type I IFN gene signature (IFNGS)³⁴ were also assessed. All efficacy and IFNGS measures were assessed from Wk 52 to end of follow-up (Wk 60); safety was assessed for 12 wks after the final study dose at Wk 48 or upon study discontinuation. The 21-gene type I IFN gene signature (IFNGS) was assessed over 8 weeks through to 60 weeks. Safety (adverse [AEs]) was evaluated over 12 weeks from Week 48 though to Week 60 or upon study discontinuation.

### 8.3 Results

Of 305 patients randomized in MUSE, 229 completed the last study visit (Wk 52): 86, 75, and 68 from the anifrolumab 300-mg, 1000-mg, and placebo groups, respectively. From Wk 52 to Wk 60, IFNGS expression increased more rapidly in the anifrolumab 300-mg group (mean neutralization ratio: 55.6% to −81.8%) vs the 1000-mg group (71.7% to 31.9%), with negligible changes in the placebo group (−59.2% to −62.6%) (FIG. 2B)

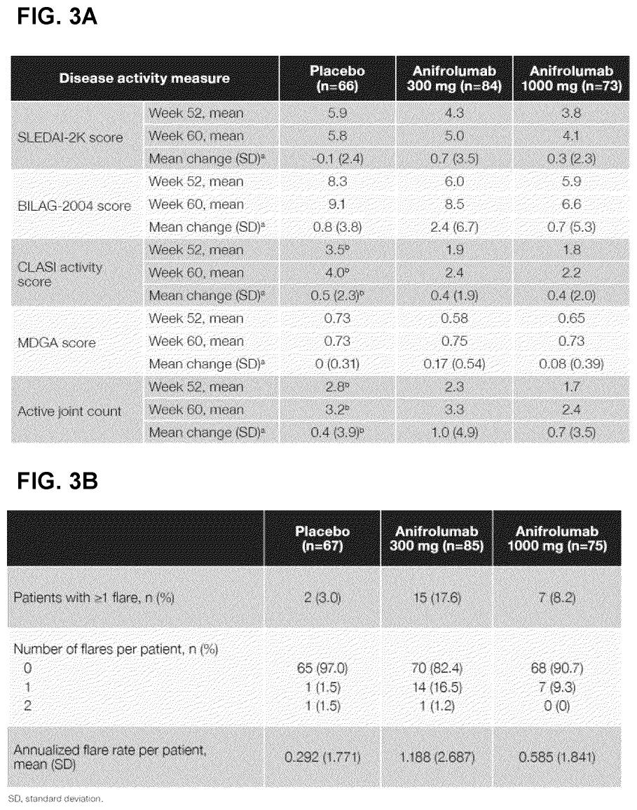

From Wk 52 to the end of the follow-up period (Wk 60), mean global SLEDAI-2K scores increased in patients coming off anifrolumab 300 mg (4.3 to 5.0 [mean change: 0.7]) and 1000 mg (3.8 to 4.1 [0.3]) but not for the placebo group (5.9 to 5.8 [−0.1]) (FIG. 3A). A similar trend was observed in mean global BILAG-2004 scores in patients coming off anifrolumab 300 mg (6.0 to 8.5 [2.4]) vs placebo (8.3 to 9.1 [0.8]) (FIG. 3A).

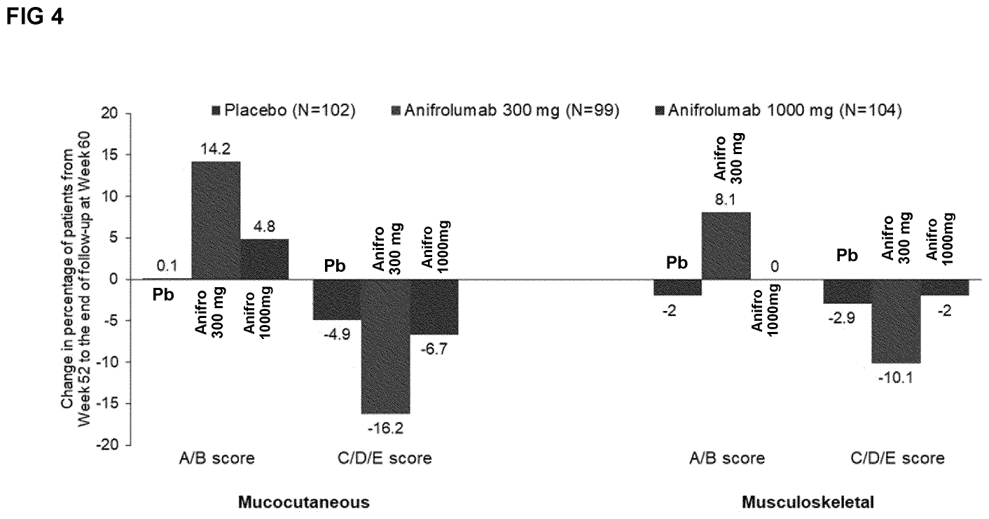

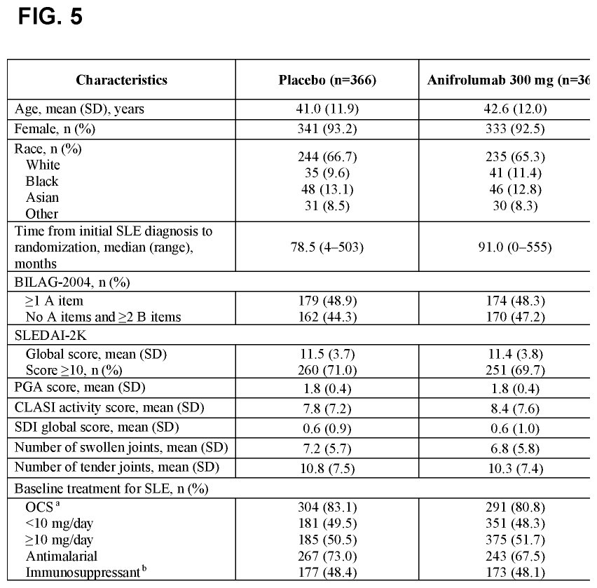

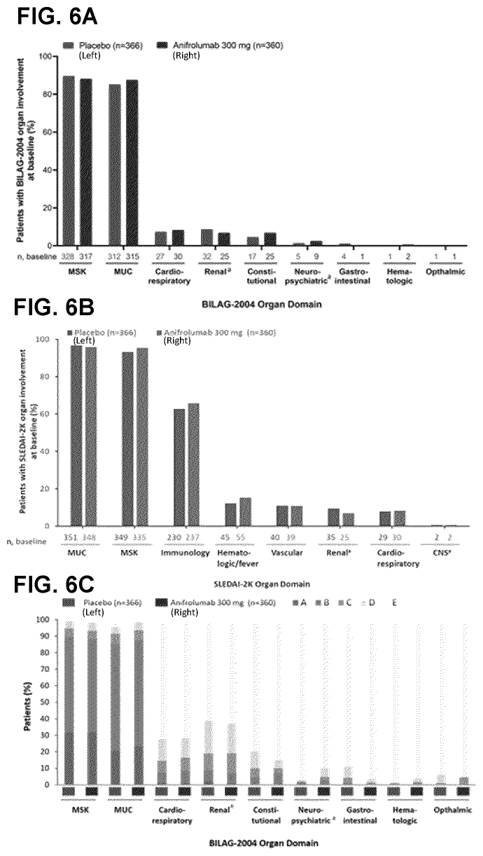

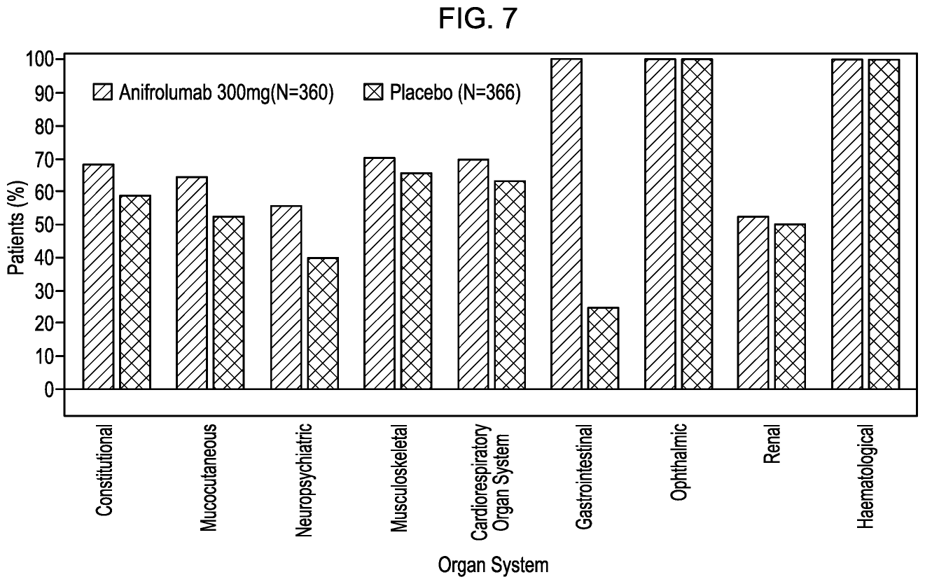

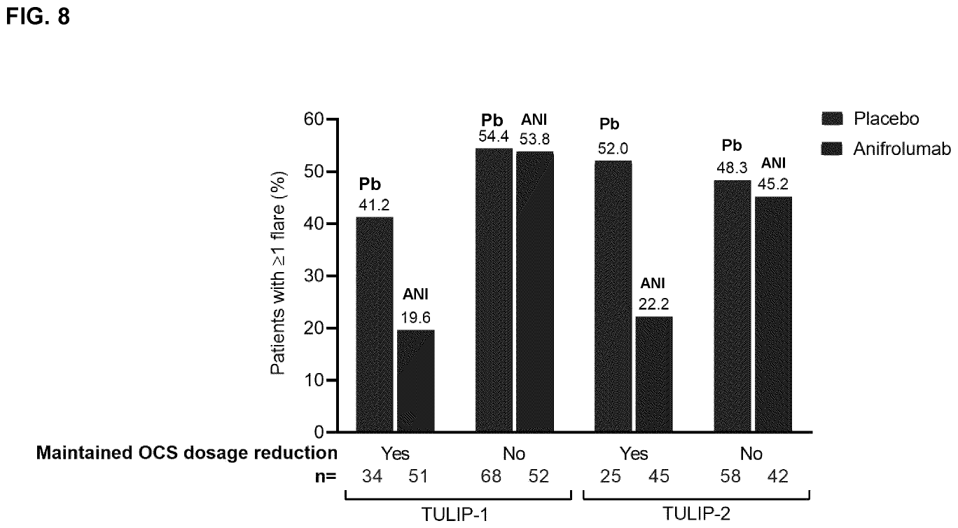

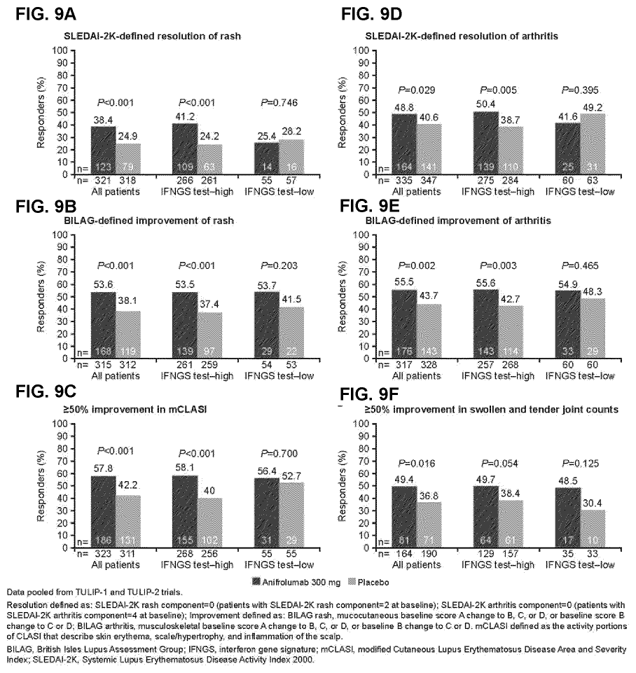

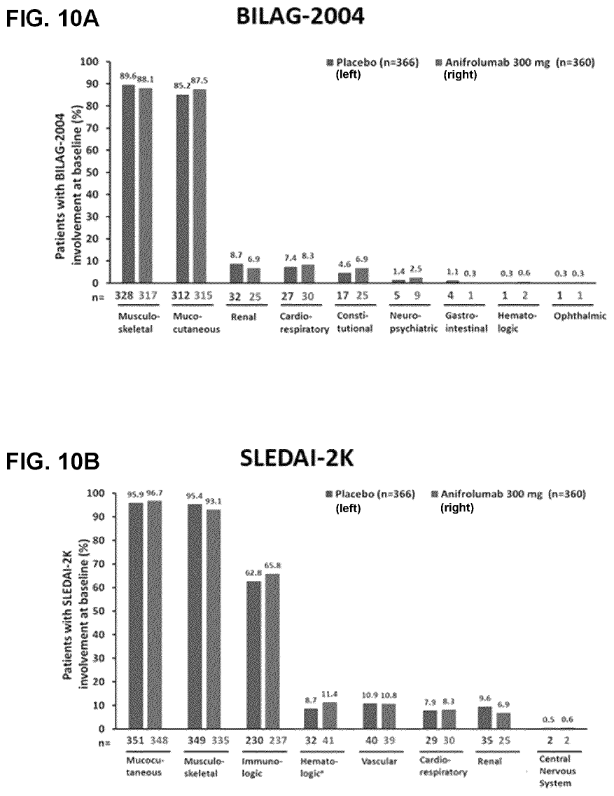

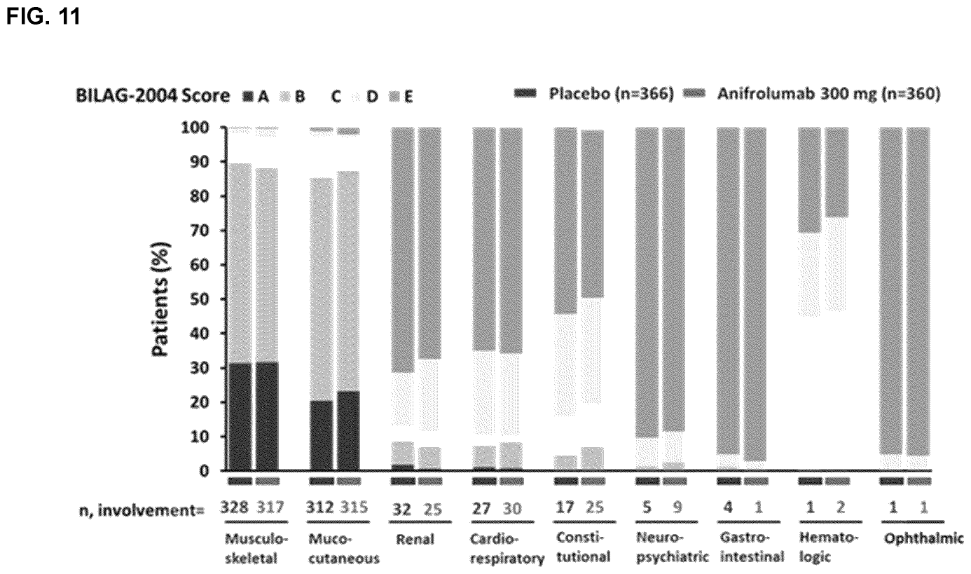

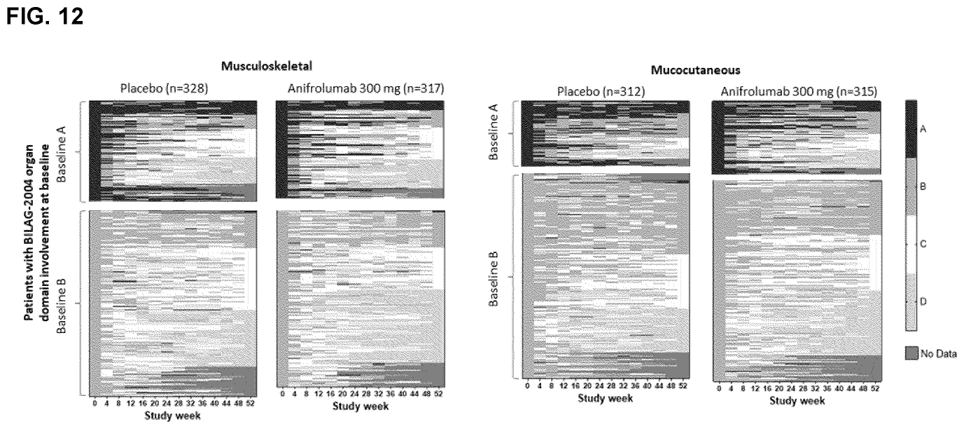

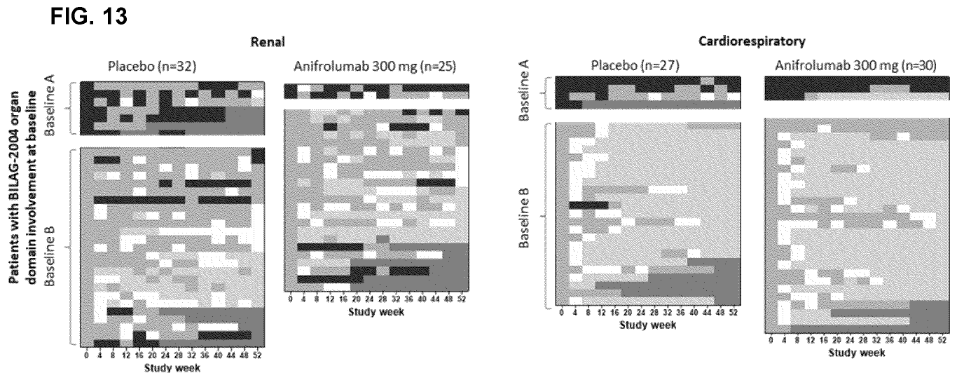

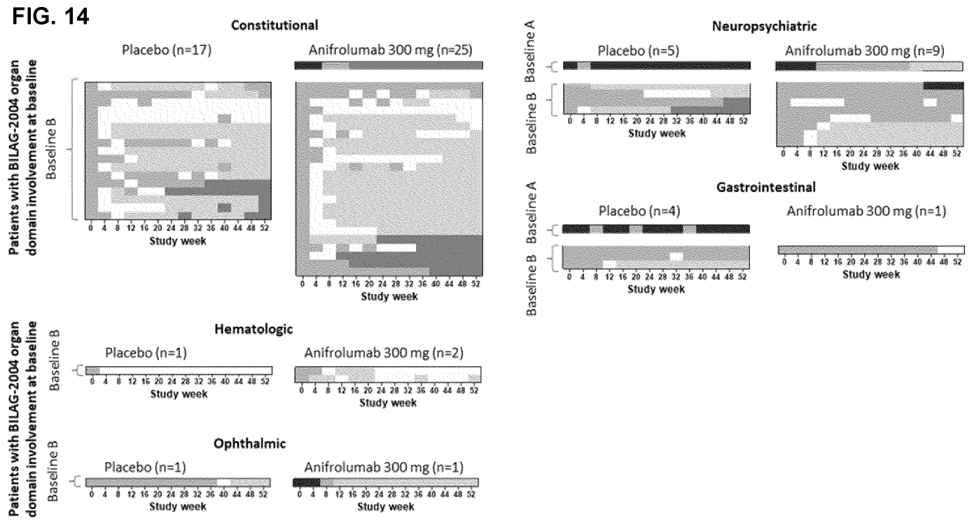

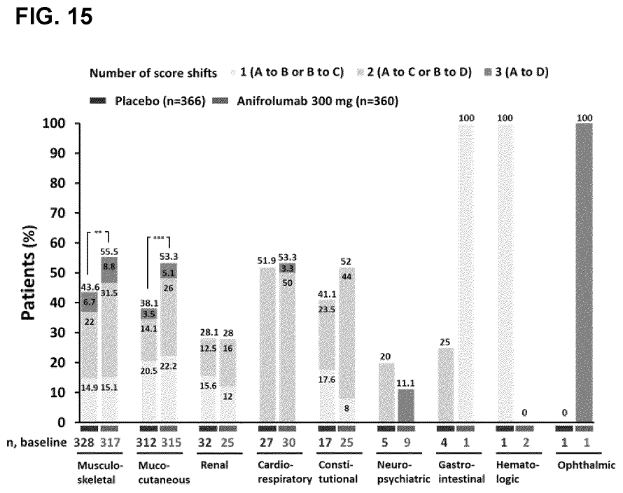

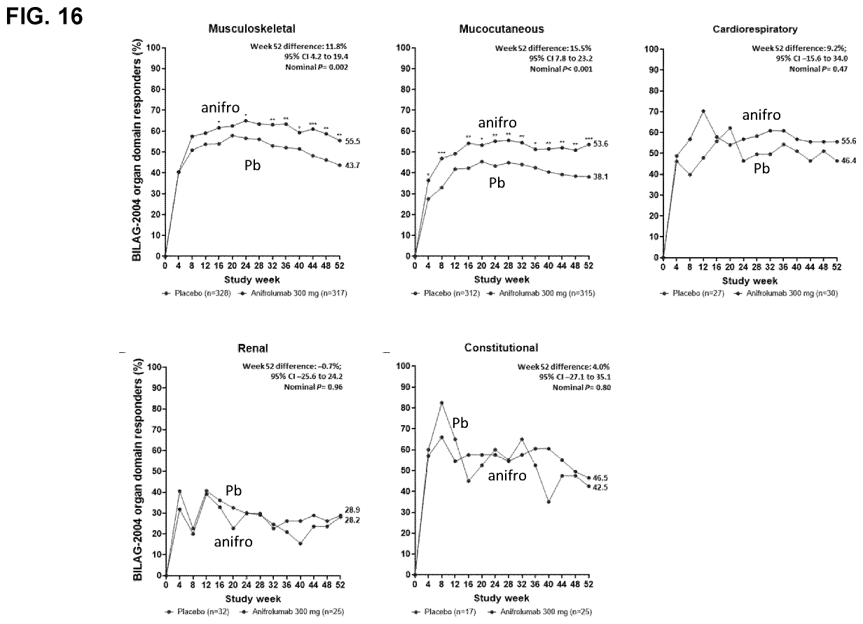

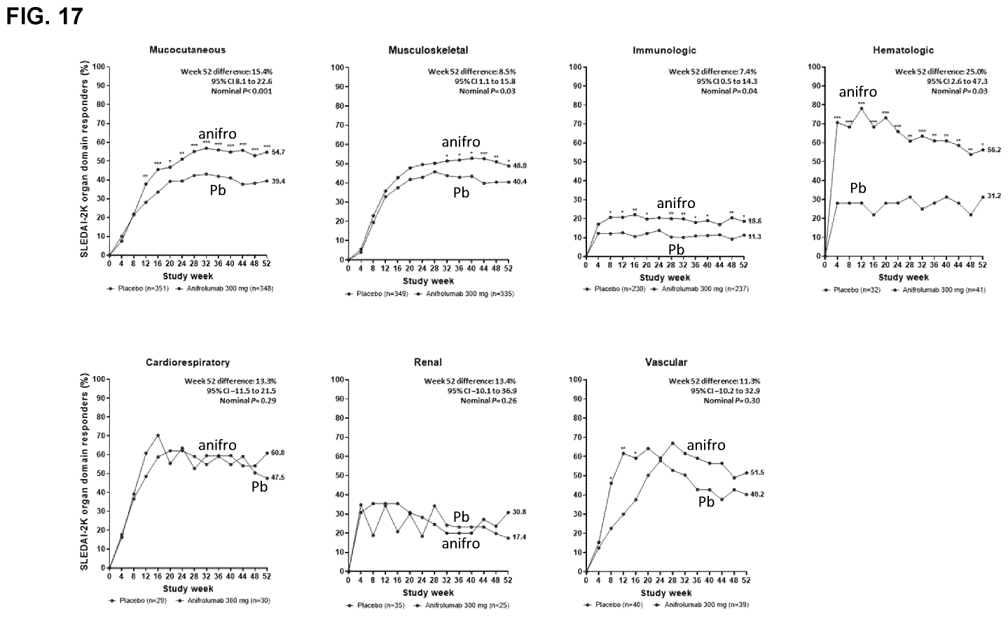

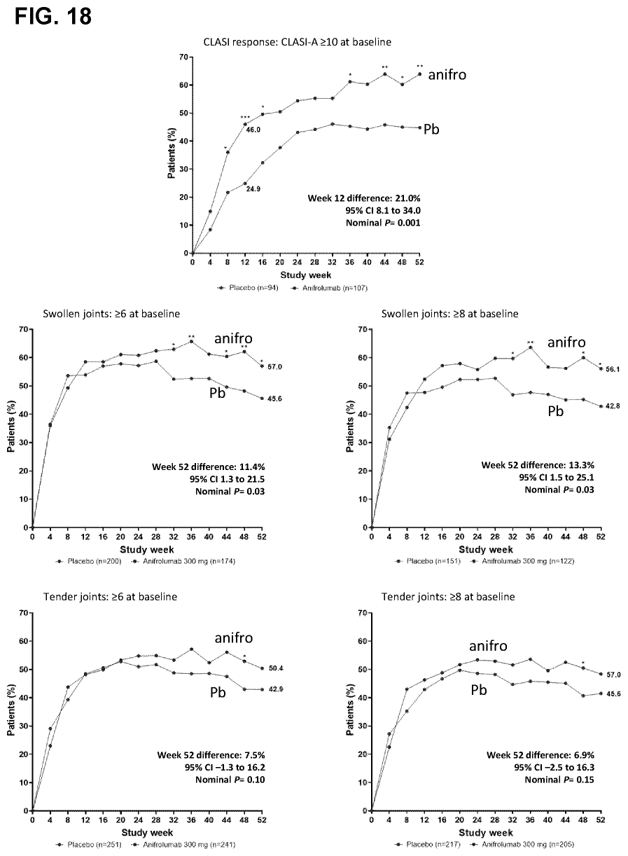

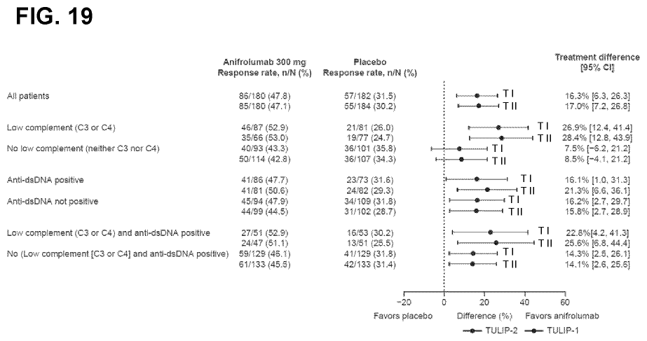

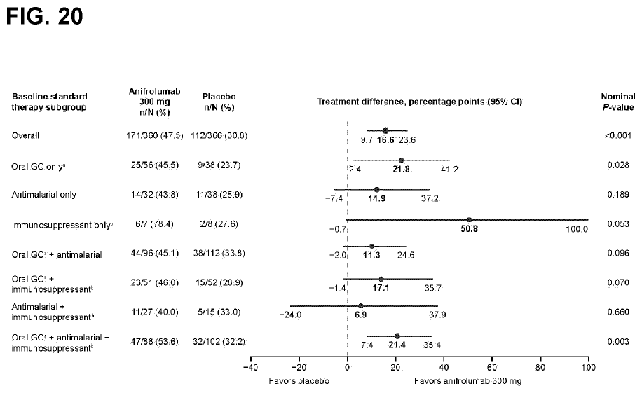

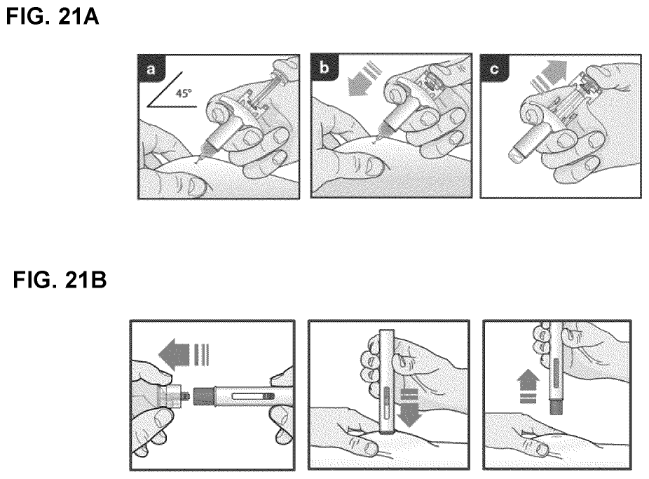

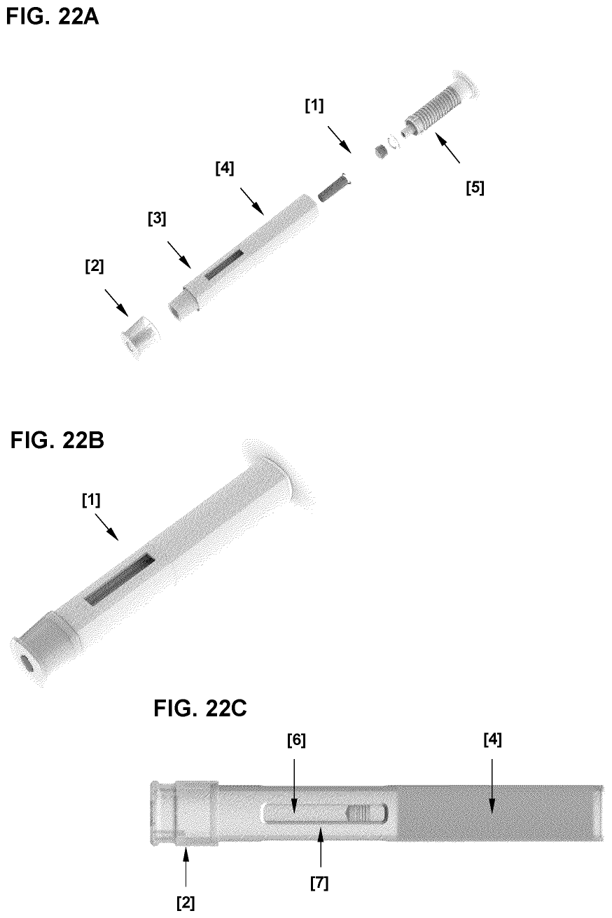

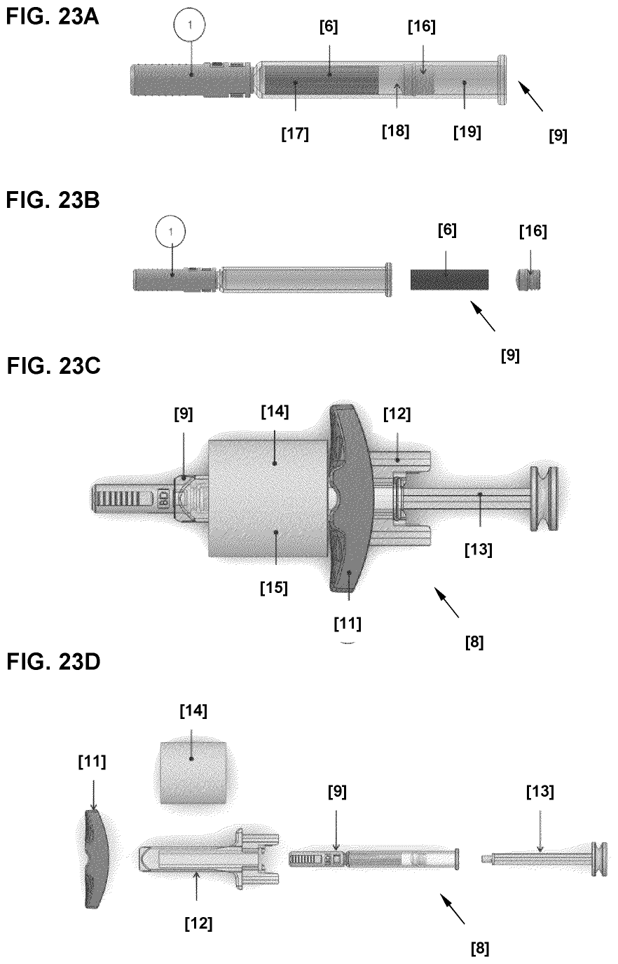

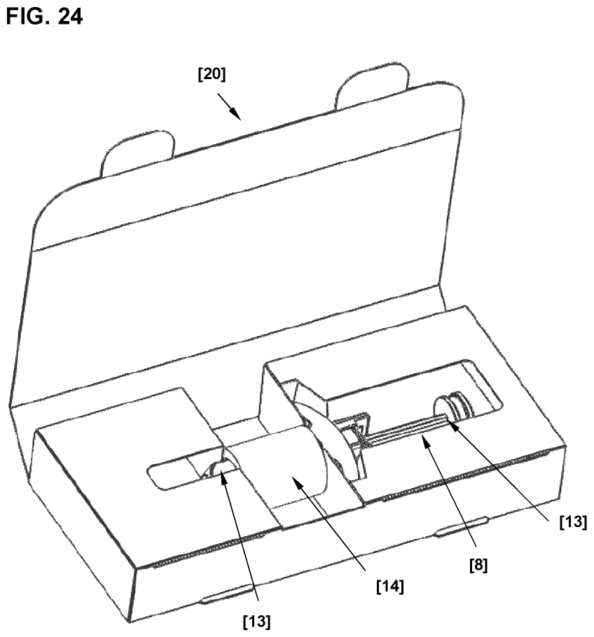

Mucocutaneous was the most frequent organ system associated with worsening in patients ceasing anifrolumab, with shifts in the percentages of patients with BILAG C/D/E scores to BILAG A/B scores; similar trends were also observed in the musculoskeletal organ system. Worsening was most frequent in the mucocutaneous domain in patients coming off anifrolumab, with shifts in the percentages of patients with BILAG-2004 C/D/E to A/B scores (FIG. 4); similar trends were also observed in the musculoskeletal domain. Overall, 15.2% and 6.7% of patients coming off anifrolumab 300 or 1000 mg, respectively, had ≥1 flare in the follow-up period vs 2.0% with placebo.

Mean Cutaneous Lupus Erythematosus Disease Area and Severity Index (CLASI) scores increased slightly from Wk 52 to Wk 60 across the anifrolumab 300-mg, 1000-mg, and placebo groups (from 1.9 to 2.4, 1.8 to 2.2, and 3.5 to 4.0, respectively) (FIG. 3A).

From Wk 52 to Wk 60, IFNGS expression increased more rapidly in the anifrolumab 300-mg group (mean neutralization ratio: 55.6% to −81.8%) vs the 1000-mg group (71.7% to 31.9%), with negligible changes in the placebo group (−59.2% to −62.6%). AEs during the 12-wk follow-up period were similar between the anifrolumab 300-mg and 1000-mg vs placebo groups (≥1 AE: 29.3% and 26.7% vs 24.8%; ≥1 serious AE: 3.0% and 3.8% vs 5.0%). Disease activity, measured using MDGA score, increased between Week 52 and Week 60 in both anifrolumab 300-mg and 1000-mg groups; there was no change in the placebo group (FIG. 3A). Active joint counts increased slightly from Week 52 to Week 60 across the anifrolumab 300-mg, anifrolumab 1000-mg, and placebo groups (FIG. 3A). Overall, more patients ceasing treatment of anifrolumab 300 or 1000 mg had ≥1 BILAG flare from Week 52 through Week 60 versus placebo (FIG. 3B).

### 8.4 CONCLUSION

There was a notable trend toward worsening in disease activity in patients coming off anifrolumab vs placebo using SLEDAI-2K and BILAG-2004. This was associated with a rebound in IFNGS in patients previously treated with anifrolumab, an effect more apparent with 300 vs 1000 mg.

### 9 Example 4: Flare Assessments by Organ Domain and OCS Taper in Patients With Active SLE Treated With Anifrolumab in 2 Phase 3 Trials

### 9.1 Introduction

SLE disease flares and SLE treatment with oral corticosteroids (OCS) are associated with organ damage accrual. Patients with SLE who received anifrolumab, a monoclonal antibody to the type I interferon receptor subunit 1, had lower flare rates and were able to taper OCS dosage versus placebo in the phase 3 trials, TULIP-1 (NCT02446912) and TULIP-2 (NCT02446899). The inventors evaluated the effect of anifrolumab treatment on flares by organ domain and in relation to OCS taper in the TULIP trials.

### 9.2 Methods

The randomized, double-blind, placebo-controlled TULIP-1 and TULIP-2 trials evaluated the efficacy and safety of anifrolumab (300 mg IV every 4 weeks for 48 weeks, primary endpoint at Week 52) in patients with moderately to severely active SLE despite standard-of-care treatment. Flares were defined as ≥1 new BILAG-2004 A or ≥2 new BILAG-2004 B domain scores versus the prior visit. An OCS tapering attempt to 57.5 mg/day was required between Weeks 8 and 40 for patients receiving baseline OCS ≥10 mg/day. Maintained OCS dosage reduction was defined as OCS dosage of ≤7.5 mg/day achieved by Week 40 and maintained to Week 52. TULIP-1 and −2 were analysed separately using restricted medication rules per the TULIP-2 protocol, and data from both trials were pooled. The inventors analysed flares descriptively by organ domain and in patients on OCS 210 mg/day at baseline with maintained OCS reduction.

### 9.3 Results

Data were pooled for 726 patients; 360 received anifrolumab 300 mg (180 patients in each trial) and 366 received placebo (184 and 182 patients in TULIP-1 and TULIP-2, respectively). Baseline patient demographics and treatment characteristics were comparable between treatment groups (FIG. 5). In the anifrolumab and placebo arms, the majority of patients had 21 BILAG A organ domain score (48.3% and 48.9%, respectively) or no A items and 22 B items (47.2% and 44.3%) (FIG. 5). The mean (SD) SLEDAI-2K score was 11.4 (3.8) and 11.5 (3.7) in the anifrolumab and placebo groups, respectively. The most commonly affected organ domains at baseline were mucocutaneous (BILAG-2004 86.4%, n=627; SLEDAI-2K 96.3%, n=699) musculoskeletal (BILAG-2004 88.8%, n=645; SLEDAI-2K 94.2%, n=684), and immunologic (SLEDAI-2K 64.3%, n=467) (FIG. 6A-C); central nervous system (CNS)/neuropsychiatric and renal involvement were relatively uncommon at baseline for both BILAG-2004 (<3%, neuropsychiatric; <8%, renal) and SLEDAI-2K (<0.6%, CNS; <10%, renal) because of the exclusion of patients with severe active lupus nephritis or severe active CNS manifestations. Baseline organ domain involvement assessed by BILAG-2004 and SLEDAI-2K was similar between treatment groups (FIG. 6A-C).

At Week 52, a greater number of patients treated with anifrolumab vs placebo had improvements in the BILAG-2004 mucocutaneous and musculoskeletal domain scores. Improvements were also observed in the majority of less frequently affected domains (FIG. 7).

In total, 360 patients received anifrolumab (TULIP-1, n=180; TULIP-2, n=180) and 366 received placebo (TULIP-1, n=184; TULIP-2, n=182). Overall, fewer patients had 21 flare with anifrolumab (33.6%, n=121) versus placebo (42.9%, n=157). Flares occurred most frequently in the mucocutaneous, musculoskeletal, and renal domains in both treatment groups; across all 3 domains, fewer patients experienced 21 flare with anifrolumab (22.8%, 19.4%, and 5.0%) versus placebo (26.8%, 25.4%, and 7.4%) (Table 9-1).

TABLE 9-1
Number of Flares by Organ Domain in Pooled TULIP-1 and TULIP-2 Data.

| | Overall, n (%) | | Mucocutaneous, n (%) | | Musculoskeletal, n (%) | | Renal, n (%) | |
|---|---|---|---|---|---|---|---|---|
| | | Anifrolumab | | Anifrolumab | | Anifrolumab | | Anifrolumab |
| Number | Placebo | 300 mg | Placebo | 300 mg | Placebo | 300 mg | Placebo | 300 mg |
| of flares | (n = 366) | (n = 360) | (n = 366) | (n = 360) | (n = 366) | (n = 360) | (n = 366) | (n = 360) |
| 0 | 209 (57.1) | 239 (66.4) | 268 (73.2) | 278 (77.2) | 273 (74.6) | 290 (80.6) | 339 (92.6) | 342 (95.0) |
| ≥1 | 157 (42.9) | 121 (33.6) | 98 (26.8) | 82 (22.8) | 93 (25.4) | 70 (19.4) | 27 (7.4) | 18 (5.0) |
| 1 | 89 (24.3) | 74 (20.6) | 68 (18.6) | 62 (17.2) | 67 (18.3) | 48 (13.3) | 17 (4.6) | 10 (2.8) |
| 2 | 49 (13.4) | 28 (7.8) | 26 (7.1) | 18 (5.0) | 21 (5.7) | 14 (3.9) | 7 (1.9) | 5 (1.4) |
| ≥3 | 19 (5.2) | 19 (5.3) | 4 (1.1) | 2 (0.6) | 5 (1.4) | 8 (2.2) | 3 (0.8) | 3 (0.8) |

BILAG, British Isles Lupus Assessment Group.
A flare in the overall group is defined as ≥1 new BILAG-2004 A or ≥2 new BILAG-2004 B items compared with the prior visit. A flare in a BILAG organ domain is present if the respective organ is associated with a flare. Data are presented for organ domains associated with ≥1 flare in ≥5% of patients in the anifrolumab group.

Fewer patients with maintained OCS reduction experienced ≥1 flare with anifrolumab (TULIP-1: 19.6%; TULIP-2: 22.2%) versus placebo (TULIP-1: 41.2%; TULIP-2: 52.0%) (FIG. 8). Similar percentages of patients without maintained OCS reduction experienced ≥1 flare with anifrolumab (TULIP-1: 53.8%; TULIP-2: 45.2%) versus placebo (TULIP-1: 54.4%; TULIP-2: 48.3%).

### 9.4 CONCLUSIONS

In the phase 3 TULIP-1 and TULIP-2 trials, fewer patients experienced flares across the 3 most frequently affected organ domains (mucocutaneous, musculoskeletal, and renal) with anifrolumab versus placebo. Anifrolumab was surprisingly associated with a ≥2-fold reduction in flares in patients with maintained OCS dosage reduction versus placebo. TULIP data support the capacity of anifrolumab to reduce SLE flares during OCS taper, an important attribute for the long-term management of patients with SLE. Results of the TULIP-1 and TULIP-2 trials previously demonstrated that patients treated with anifrolumab had higher BICLA responder rates. Both BILAG and SLEDAI are incorporated into the BICLA index. However, BILAG was used for evaluating improving and worsening, and SLEDAI-2K was only used for worsening. Evaluation of individual organ domains as assessed by BILAG-2004 and SLEDAI-2K demonstrated that anifrolumab treatment, compared with placebo, was associated with improvement in the most frequently affected organ domains (mucocutaneous, musculoskeletal).

### 10 Example 5: Anifrolumab Effects on Rash and Arthritis for Patients With SLE, and Impact of Interferon Signal in Pooled Data From Phase 3 Trials

### 10.1 Background

Treatment with anifrolumab is associated with clinical improvements in mucocutaneous and musculoskeletal disease activity versus placebo in patients with SLE in the phase 2 MUSE trial (NCT01438489) and the phase 3 TULIP trials (FIG. 4 and FIG. 7). The inventors examined symptom-targeted effects of biomarker-defined subsets.

### 10.2 Aim

To evaluate the effect of anifrolumab on rash and arthritis, and the impact of IFN gene signature (IFNGS) status in patients with SLE using disease measures of different sensitivity in pooled data from the phase 3 TULIP trials.

### 10.3 Methods

TULIP-1 (NCT02446912) and TULIP-2 (NCT02446899) were placebo-controlled, 52-week trials of intravenous anifrolumab administered every 4 weeks in patients with moderate to severe SLE. In this post hoc analysis, outcomes of rash and arthritis were evaluated using the mucocutaneous and musculoskeletal domains of the Systemic Lupus Erythematosus Disease Activity Index 2000 (SLEDAI-2K) (stringent measure) and the British Isles Lupus Assessment Group (BILAG) index (more sensitive measure capturing partial improvements). Improvements in rash, using the modified Cutaneous Lupus Erythematosus Disease Area and Severity Index (mCLASI) score, and in arthritis, assessed by tender and swollen joint counts, were also evaluated.

### 10.4 Results

In pooled data from TULIP-1 and TULIP-2, 600 patients (anifrolumab 300 mg, n=298; placebo, n=302) were classified as IFNGS test-high and 126 patients (anifrolumab, n=62; placebo, n=64) as IFNGS test-low. Overall, more anifrolumab-treated patients versus placebo achieved SLEDAI-2K-defined complete resolution of rash (difference 13.5%, nominal P<0.001) (FIG. 9A). The more sensitive measure, BILAG, which required an improvement of ≥1 grade, showed a benefit of anifrolumab over placebo for rash (difference 15.5%, nominal P<0.001); results were comparable in the IFNGS test-high subset (SLEDAI-2K: difference 17%, nominal P<0.001; BILAG: difference 16.1%, nominal P<0.001) (FIG. 9B). In IFNGS test-low patients, there was a trend towards anifrolumab-associated rash improvement. Improvements of ≥50% from baseline to Week 52, defined by mCLASI, in patients with baseline mCLASI activity scores ≥0, were more frequent with anifrolumab versus placebo (difference 15.6%, nominal P<0.001) (FIG. 9C). Overall, more anifrolumab-treated patients versus placebo achieved SLEDAI-2K-defined complete resolution in arthritis (difference 8.2%, nominal P=0.029) (FIG. 9D). This was also seen using the BILAG-defined improvement in arthritis (difference 11.8%, nominal P=0.002) (FIG. 9E). There were comparable results in the IFNGS test-high subset (SLEDAI-2K: difference 11.7%, nominal P=0.005; BILAG: difference 12.9%, nominal P=0.003) but not in IFNGS test-low patients (FIG. 9D and FIG. 9E). Anifrolumab's efficacy was further confirmed by a ≥50% improvement in swollen and tender joint counts, in patients with ≥6 at baseline (difference 12.6%, nominal P=0.016); the effect was comparable in IFNGS test-high and test-low patients (FIG. 9F).

### 10.5 Conclusions

In pooled data from TULIP-1 and TULIP-2, anifrolumab treatment was associated with improvements versus placebo in rash and arthritis using measures of different stringency.

### 11 Example 6: Effects of Anifrolumab on Renal Disease in Patients with SLE

### 11.1 Background

The type I interferon (IFN) receptor antibody anifrolumab has shown efficacy in patients with systemic lupus erythematosus (SLE) in the phase 3 TULIP-1 and TULIP-2 trials, which excluded patients with severe active lupus nephritis (LN)^{28,30}.

### 11.2 Aim

Pooled TULIP data were analysed post hoc to assess baseline characteristics of patients with and without renal involvement, and to evaluate the effects of anifrolumab on renal disease.

### 11.3 Methods

TULIP-1 (NCT02446912) and TULIP-2 (NCT02446899) were randomized, placebo-controlled, 52-week (W) trials of intravenous anifrolumab every 4 weeks in patients with moderate to severe SLE despite standard therapy. Renal involvement at baseline was defined as any of the following: BILAG-2004 renal score A-C; SLE Disease Activity Index 2000 (SLEDAI-2K) renal score ≥0; urine protein-creatinine ratio (UPCR) ≥0.5 mg/mg. Baseline characteristics were evaluated in patients with and without renal involvement, and the following endpoints were compared for the anifrolumab 300 mg and placebo groups: cumulative UPCR (area under the curve, AUC) through W52; percentage of patients with UPCR ≥0.5 mg/mg at baseline who improved to UPCR 50.5 mg/mg by W52; cumulative glucocorticoid (GC) use (AUC) through W52; percentage changes in complement C3 and C4 from baseline to W52; and percentage of patients with renal flares (new BILAG-2004 A/B renal score vs prior visit).

### 11.4 Results

Of the 726 patients in TULIP-1/TULIP-2 (anifrolumab, n=360; placebo, n=366), 99 had renal involvement at baseline (anifrolumab, n=45; placebo, n=54), 57 of whom had UPCR ≥0.5 mg/mg (anifrolumab, n=24; placebo, n=33). Compared with patients without renal involvement, patients with renal involvement had a mean age of 37.8 vs 42.4 years, and were more likely to be male (14.1% vs 6.1%), Asian (16.2% vs 9.6%), IFN gene signature test-high (89.9% vs 81.5%), anti-dsDNA positive (69.7% vs 40.4%), have a SLEDAI-2K score ≥10 (91.9% vs 68.4%), and be receiving GC ≥10 mg/day (67.7% vs 49.1%) or mycophenolate (26.3% vs 11.5%) at baseline. Among patients with baseline renal involvement, anifrolumab treatment was associated with a numerically greater improvement vs placebo in cumulative UPCR (AUC) through W52 (mean difference [SE]:−54.1 [54.26]) (Table 11-1). Numerically more patients improved from UPCR ≥0.5 mg/mg at baseline to 50.5 mg/mg at W52 with anifrolumab vs placebo (difference [SE], 4.9% [13.3]). Cumulative GC use (AUC) through W52 was lower with anifrolumab vs placebo among patients with baseline renal involvement (LS mean difference [SE]:−210.3 mg [332.6]). There were numerically greater improvements in C3 and C4 from baseline to W52 with anifrolumab vs placebo among patients with baseline renal disease (Table 11-1). Among all TULIP patients, fewer had ≥1 renal flare with anifrolumab vs placebo (5.0% vs 7.4%).

### Table 11-1: Renal Endpoints in TULIP-1 and TULIP-2

^{a}Patients with renal involvement at baseline; analysis of covariance. ^{b}Stratified Cochran-Mantel-Haenszel approach. ° Patients with renal involvement and abnormal C3 or C4 at baseline. AUC, area under the curve; LS, least squares; UPCR, urine protein-creatinine ratio; SE, standard error.

### 11.5 CONCLUSIONS

TULIP data indicate renal benefit with anifrolumab in patients with SLE with stable/inactive renal disease.

### 12 Example 7: Efficacy of Anifrolumab Across Organ Domains in Patients with Moderate to Severe SLE in Pooled Data from the TULIP-1 and TULIP-2 Trials

### 12.1 Introduction

The similarity in design of the TULIP-1 and TULIP-2 trials facilitated pooling of data for assessment of individual organ systems with greater statistical power than possible with individual trials alone. In this post hoc analysis of pooled data from the TULIP-1 and TULIP-2 trials, we assessed the effects of anifrolumab on individual SLE organ domain disease activity.

### 12.2 Methods

### 12.2.1 Patients and Study Design

| Endpoint, | | Anifrolumab |
|---|---|---|
| from baseline to W 52 | Placebo | 300 mg |

UPCR AUCa

| n | 54 | | 45 | |
|---|---|---|---|---|
| LS mean (SE) | 271.8 | (54.8) | 217.7 | (60.0) |

| | |
|---|---|
| Mean difference (SE) | −54.1 (54.3) |

Improvement from >0.5 to ≤0.5 mg/mg UPCRb

| | | |
|---|---|---|
| n | 33 | 24 |
| Patients with improvement (%) | 36.3 | 41.2 |

| | |
|---|---|
| Difference, % (SE) | 4.9 (13.3) |

Glucocorticoid AUCa

| n | 54 | | 45 | |
|---|---|---|---|---|
| LS mean (SE) | 3524.5 | (339.0) | 3314.2 | (365.2) |

| | |
|---|---|
| Mean difference (SE) | −210.3 (332.6) |

Percentage change in C3 and C4c

| C3 | | | | |
|---|---|---|---|---|
| n | 31 | | 21 | |
| Mean (SE) | 20.3 | (6.2) | 26.6 | (5.0) |
| C4 | | | | |
| n | 19 | | 14 | |
| Mean (SE) | 29.1 | (12.0) | 38.7 | (13.8) |

This was a post hoc analysis of pooled data from the 52-week TULIP-1 and TULIP-2 trials, in which patients who had moderate to severe SLE despite standard therapy with oral glucocorticoids, antimalarials, and/or immunosuppressants were randomized to receive anifrolumab 300 mg or placebo intravenously every 4 weeks for 48 weeks.

The study design and methods have been described in detail previously^{28,30}. In brief, all patients were aged 18 to 70 years and fulfilled the American College of Rheumatology classification criteria for SLE. Patients with active severe neuropsychiatric SLE or severe lupus nephritis were excluded. Mandatory attempts to taper oral glucocorticoids to 57.5 mg/day between Week 8 and Week 40 were required for patients receiving prednisone or equivalent ≥10 mg/day at baseline; tapering was also permitted for patients receiving lower doses at baseline. In all patients, glucocorticoid doses were required to be stable from Week 40 through Week 52.

### 12.2.2 Study Endpoints and Assessments

Organ domain involvement was assessed using BILAG-200417 and SLEDAI-2K.18 BILAG-2004 response was defined as a reduction from A (severe disease) at baseline to B (moderate), C (mild), or D (no current disease), or from B at baseline to C or D. The proportions of patients who improved 1 step (eg, from A to B or B to C), 2 steps (eg, from A to C or B to D), and up to 3 steps (ie, from A to D) in a given organ domain from baseline to Week 52 were evaluated. SLEDAI-2K improvement was defined as a reduction in domain scores in patients with baseline scores >0. For both BILAG-2004 and SLEDAI-2K, patients who were treated with restricted medication beyond protocol-allowed thresholds or who discontinued investigational product were classified as nonresponders.

Skin and joint disease were further assessed using the Cutaneous Lupus Erythematosus Disease Area and Severity Index (CLASI) activity score (CLASI-A)³⁵ and swollen and 28 swollen and tender joint counts, respectively. CLASI response was defined as ≥50% reduction in CLASI-A among patients with baseline CLASI-A ≥10. Improvement in joint counts was defined as a reduction of ≥50% from baseline in counts of swollen or tender joints in patients with ≥6 swollen and ≥6 tender joints at baseline; a second analysis included those with 8 swollen and ≥8 tender joints at baseline.

In addition to changes in mean hematologic and serologic values, the percentages of patients with abnormal (low or high) values at baseline who converted to normal values at Week 52 were evaluated. Patients who discontinued study treatments or had missing Week 52 data were assumed not to have normalized.

### 12.2.3 STATISTICAL ANALYSES

The similar TULIP-1 and TULIP-2 trial designs allowed for the results to be pooled. BILAG-2004 and SLEDAI-2K organ domain responder rates, SLEDAI-2K organ domain responders overtime, CLASI-A responders over time, and 50% reductions in joint counts from baseline were calculated using a stratified Cochran-Mantel-Haenszel approach, with stratification factors (matching those in the TULIP studies) of SLEDAI-2K score at screening, type I IFN gene signature test status at screening, and Day 1 oral glucocorticoid dose. The reported 2-sided P-values and 95% confidence intervals (CIs) are based on this approach. All reported P-values are nominal. For assessment of pooled TULIP data, TULIP-1 data were analysed according to the TULIP-2-revised restricted medication analytic rules. Missing data were imputed using the last observation carried forward for the first visit with missing data; subsequent visits with missing data were not imputed.

### 12.3 Results 12.3.1 Baseline Characteristics

Data were pooled for 726 patients; 360 received anifrolumab 300 mg (180 patients in each trial), and 366 received placebo (184 and 182 patients in TULIP-1 and TULIP-2, respectively). Baseline demographics and background treatment for SLE were comparable between groups (Table 12-1).

TABLE 12-1
Baseline patient demographics, disease characteristics,
and SLE medications of patients enrolled in
TULIP-1 and TULIP-2 (pooled data)

| | | | Anifrolumab | |
|---|---|---|---|---|
| | Placebo | | 300 mg | |
| Characteristics | (n = 366) | | (n = 360) | |
| Age, mean (SD), years | 41.0 | (11.9) | 42.6 | (12.0) |
| Female, n (%) | 341 | (93.2) | 333 | (92.5) |

Race, n (%)

| | | | | |
|---|---|---|---|---|
| White | 244 | (66.7) | 235 | (65.3) |
| Black | 48 | (13.1) | 46 | (12.8) |
| Asian | 35 | (9.6) | 41 | (11.4) |
| Other | 31 | (8.5) | 30 | (8.3) |
| Time from initial SLE diagnosis to | 78.5 | (4-503) | 91.0 | (0-555) |
| randomization, median (range), months | | | | |

BILAG-2004, n (%)

| | | | | |
|---|---|---|---|---|
| ≥1 A item | 179 | (48.9) | 174 | (48.3) |
| No A items and ≥2 B items | 162 | (44.3) | 170 | (47.2) |

SLEDAI-2K

| | | | | |
|---|---|---|---|---|
| Mean (SD) | 11.5 | (3.7) | 11.4 | (3.8) |
| ≥10, n (%) | 266 | (72.7) | 254 | (70.6) |
| PGA, mean (SD) | 1.8 | (0.4) | 1.8 | (0.4) |
| CLASI-A | 7.8 | (7.2) | 8.4 | (7.6) |
| Mean (SD) | 7.8 | (7.2) | 8.4 | (7.6) |
| ≥10, n (%) | 4 | (25.7) | 107 | (29.7) |
| SDI, mean (SD) | 0.6 | (0.9) | 0.6 | (1.0) |
| Number of swollen joints, mean (SD) | 7.2 | (5.7) | 6.8 | (5.8) |
| Number of tender joints, mean (SD) | 10.8 | (7.5) | 10.3 | (7.4) |

Baseline treatment for SLE, n (%)

| | | | | |
|---|---|---|---|---|
| Oral glucocorticoid use^{a} | 304 | (83.1) | 291 | (80.8) |
| <10 mg/day | 181 | (49.5) | 170 | (47.2) |
| ≥10 mg/day | 185 | (50.5) | 190 | (52.8) |
| Antimalarial | 267 | (73.0) | 243 | (67.5) |
| Immunosuppressant^{b} | 177 | (48.4) | 173 | (48.1) |

BILAG-2004, British Isles Lupus Assessment Group-2004; CLASI, Cutaneous Lupus Erythematosus Disease Area and Severity Index; CLASI-A, CLASI activity score; PGA, Physician's Global Assessment; SD, standard deviation; SDI, Systemic Lupus International Collaborating Clinics/American College of Rheumatology Damage Index; SLE, systemic lupus erythematosus; SLEDAI-2K, Systemic Lupus Erythematosus Disease Activity Index 2000.
^{a}Oral glucocorticoids contains prednisone or equivalent;
^{b}Immunosuppressant: azathioprine, methotrexate, mycophenolate mofetil, mycophenolic acid, and mizoribine.

Of the 726 patients enrolled, the mean age was 41.8 years; 92.8% were women, and 66.0% were white. At baseline, 82.0% (595/726) of patients were receiving oral glucocorticoids, of whom 52.8% (190/360) of the anifrolumab group and 50.5% (1 85/366) of the placebo group were receiving ≥10 mg/day (prednisone or equivalent). Baseline disease activity levels, measured with BILAG and SLEDAI-21K, were similar between the pooled treatment arms (Table 12-1), with a mean SLEDAI-2K of approximately 11 and approximately half of all patients having at least one BILAG A domain score.

Baseline organ domain involvement assessed using BILAG-2004 and SLEDAI-2K was similar between treatment groups (FIG. 10A and FIG. 10B). The most commonly affected organ domains at baseline were mucocutaneous (BILAG-2004 86.4% [627/726]; SLEDAI-2K 96.3% [699/726]), musculoskeletal (BILAG-2004 88.8% [645/726]; SLEDAI-2K 94.2% [684/726]), and immunologic (SLEDAI-2K 64.3% [467/726]) (FIG. 10A and FIG. 10B); immunologic variables are not captured in BILAG-2004. Renal and neuropsychiatric involvement were relatively uncommon at baseline regardless of whether the assessments were conducted with BILAG-2004 or SLEDAI-2K. In the most commonly affected BILAG-2004 domains, musculoskeletal and mucocutaneous, the majority of patients had severe or moderate disease activity at baseline as shown by the overall frequency of BILAG A (musculoskeletal 31.5% [229/726], mucocutaneous 21.9% [159/726]) or BILAG B (musculoskeletal 57.3% [416/726]; mucocutaneous 64.5% [468/726]) scores. BILAG organ domain scores were balanced across treatment groups (FIG. 11); SLEDAI-2K cannot discern the severity of activity within an organ domain.

### 12.3.2 Efficacy in BILAG-2004 Organ Domains, Constitutional, Hematologic, Ophthalmic

BILAG-2004 patient-level organ domain scores obtained every 4 weeks across the entire trial period are displayed using heat maps (FIG. 12, FIG. 13 and FIG. 14). At Week 52, 55.5% (176/317) of anifrolumab-treated patients achieved a BILAG-2004 musculoskeletal response compared with 43.6% (143/328) of patients receiving placebo (difference 11.9%; 95% CI 4.2, 19.4; nominal P<0.01) (FIG. 15), and 53.3% (168/315) of patients treated with anifrolumab versus 38.1% (119/312) of patients receiving placebo achieved a BILAG-2004 mucocutaneous response (difference 15.5%; 95% CI 7.8, 23.2; nominal P<0.001) (FIG. 15). Improvements favouring anifrolumab for the mucocutaneous and musculoskeletal BILAG-2004 domains were observed from Week 4 and Week 32, respectively (both P<0.05) FIG. 16). Responses in the cardiorespiratory and constitutional domains were also more frequent in patients receiving anifrolumab versus placebo (FIG. 13, FIG. 14 and FIG. 15).

FIG. 15 shows the proportions of patients with 1-3-step BILAG-2004 improvements at Week 52 compared with baseline; a greater number of steps indicates a greater improvement. Improvements of at least 2 steps (A to C or D, or B to D) were observed for more patients receiving anifrolumab compared with placebo for all BILAG-2004 domains, except gastrointestinal and hematologic, where the numbers were low at baseline.

### 12.3.3 Efficacy in SLEDAI-2K Organ Domains

At Week 52, significantly more anifrolumab-treated than placebo-receiving patients had improvements in the SLEDAI-2K organ domains most frequently affected at baseline: mucocutaneous (54.7% [190/348] vs 39.4% [138/351]; nominal P<0.001), musculoskeletal (48.8% [164/335] vs 40.4% [141/349]; nominal P<0.05), and immunologic (18.6% [44/237] vs 11.3% [26/230]; nominal P<0.05) (FIG. 17). Improvements favouring anifrolumab for the mucocutaneous and musculoskeletal SLEDAI-2K domains were observed from Week 12 and Week 32, respectively (both P<0.05). Greater proportions of patients receiving anifrolumab versus placebo had improvements at Week 52 for less frequently affected SLEDAI-2K domains: vascular, hematologic, renal, and cardiorespiratory (FIG. 17). However, apart from the hematologic domain (56.2% [23/41] vs 31.2% [10/32]; nominal P<0.05), the differences favouring anifrolumab did not reach nominal significance. Improvements in response favouring anifrolumab for the hematologic and immunologic SLEDAI-2K domains were observed from Week 4 and were maintained to Week 52 (nominal P<0.05) (FIG. 17).

### 12.3.4 Efficacy in Skin Disease and Arthritis

In the subset of patients with baseline CLASI-A ≥10 (n=201), a significantly greater percentage of anifrolumab—than placebo-treated patients attained a CLASI-A response at Week 12 (46.0% [49/107]vs 24.9% [24/94]; nominal P<0.001) (FIG. 18). This treatment effect was maintained overtime: a greater proportion of patients treated with anifrolumab than with placebo achieved a CLASI-A response at each study visit.

Among patients with ≥6 swollen joints at baseline, 57.0% (99/174) who received anifrolumab had ≥50% reduction in swollen joint count at Week 52, compared with 45.6% (92/200) of patients who received placebo (nominal P<0.05) (FIG. 18). Similarly, of patients with 6 tender joints at baseline, more in the anifrolumab group had a reduction of 250% from baseline versus the placebo group (50.4% [121/241] vs 42.9% [107/251]; nominal P=0.10) (FIG. 18). Results were similar in the subset of patients with ≥8 swollen or ≥8 tender joints at baseline, with greater reductions in swollen joint counts (56.1% [69/122] vs 42.8% [66/1251]; nominal P<0.05) and tender joint counts (48.5% [99/205] vs 41.5% [90/217]; nominal P=0.15) in anifrolumab-treated patients compared with patients receiving placebo (FIG. 18). Analysis overtime of the attainment of ≥50% reduction in swollen joint counts in patients with either ≥6 or ≥8 swollen joints demonstrated separation between anifrolumab and placebo from Week 36 (nominal P<0.05) (FIG. 18).

### 12.3.5 Laboratory Markers—Hematology and Serology

Patients in the anifrolumab and placebo groups had similar mean hematology values at baseline (Table 12-2).

TABLE 12-2
Changes in hematologic measures from baseline to Week 52

| | |
|---|---|
| Placebo | Anifrolumab 300 mg |
| (n = 365)^{a} | (n = 360) |

Hemoglobin

| | | | | |
|---|---|---|---|---|
| Baseline mean (SD), g/L | 126.0 | (15.2) | 125.0 | (14.8) |
| Change from baseline, mean (SD), g/L | −2.7 | (11.33) | 0.5 | (10.59) |
| Normalization at Week 52 in patients with abnormal | 0 | (0) | 0 | (0) |
| hemoglobin at baseline, n (%)^{b} | | | | |

Hematocrit

| | | | | |
|---|---|---|---|---|
| Baseline mean (SD) | 0.4 | (0.04) | 0.4 | (0.04) |
| Change from baseline, mean (SD) | −0.005 | (0.03) | 0.005 | (0.03) |
| Normalization at Week 52 in patients with abnormal | 0 | (0) | 0 | (0) |
| hematocrit at baseline, n (%)^{b} | | | | |

Lymphocytes

| | | | | |
|---|---|---|---|---|
| Baseline mean (SD), 10⁹/L | 1.3 | (0.6) | 1.3 | (0.6) |
| Change from baseline, mean (SD), 10⁹/L | −0.03 | (0.5) | 0.3 | (0.6) |
| Normalization at Week 52 in patients with abnormal | 11 | (3.0) | 23 | (6.4) |
| lymphocytes at baseline, n (%)^{b} | | | | |

Neutrophils

| | | | | |
|---|---|---|---|---|
| Baseline mean (SD), 10⁹/L | 4.0 | (2.1) | 3.8 | (1.8) |
| Change from baseline, mean (SD), 10⁹/L | 0.1 | (2.0) | 0.7 | (1.8) |
| Normalization at Week 52 in patients with abnormal | 0 | (0) | 1 | (0.3) |
| neutrophils at baseline, n (%)^{b} | | | | |

Platelets

| | | | | |
|---|---|---|---|---|
| Baseline mean (SD), 10⁹/L | 250.2 | (79.8) | 239.9 | (78.2) |
| Change from baseline, mean (SD), 10⁹/L | 3.2 | (49.8) | 24.3 | (58.2) |
| Normalization at Week 52 in patients with abnormal | 1 | (0.3) | 0 | (0.0) |
| platelets at baseline, n (%)^{b} | | | | |

SD, standard deviation.
^{a}1 patient was removed from the analysis after study completion;
^{b}Range of normal values for hemoglobin (>60 to <200 g/L), hematocrit (>0.18 to <0.64), lymphocytes (>0.5 to <10.0 109/L), neutrophils (>0.5 to <20.0 109/L), and platelets (>20 to <600 109/L).

At Week 52, treatment effects favouring anifrolumab versus placebo were seen for mean (SD) increase in haemoglobin (0.5 [10.59] vs−2.7 [11.33] g/L) and platelets (24.3 [58.2] vs 3.2 [49.8]×109/L). In the anifrolumab group, 6.4% (23/360) of patients with leukopenia at baseline demonstrated normalization, versus 3.0% (11/366) of patients receiving placebo.

Among patients who were anti-dsDNA positive at baseline, mean (SD) levels of anti-dsDNA antibodies decreased with anifrolumab treatment, compared with an increase for placebo (−25.0 [238.4] vs 28.0 [498.5] U/mL; Table 3). Accordingly, 7.8% (13/167) of patients receiving anifrolumab versus 5.8% (9/155) of patients receiving placebo converted to anti-dsDNA negative by Week 52 (Table 12-3).

At Week 52, greater improvements from baseline in mean (SD) complement C3 levels were observed with anifrolumab (0.13 [0.18]) versus placebo (0.04 [0.16] U/mL) (Table 12-3). In patients with low C3 at baseline, normalization was observed in 16.2% (21/130) of anifrolumab-treated and 9.5% (13/137) of placebo-treated patients. Similarly, normalization of low baseline C4 occurred in more patients receiving anifrolumab versus placebo (22.6% [19/84] vs 7.1% [6/85]).

TABLE 12-3
Change in laboratory markers from baseline to Week 52

| | |
|---|---|
| Placebo | Anifrolumab 300 mg |
| (n = 366) | (n = 360) |

Anti-dsDNA^{a, b}

| | | | | |
|---|---|---|---|---|
| Anti-dsDNA positive at baseline, n (%) | 155 | (42.3) | 167 | (43.4) |
| Mean (SD), U/mL | 211.95 | (549.65) | 129.34 | (261.40) |
| Change from baseline, mean (SD), U/mL | 27.96 | (498.47) | −24.98 | (238.39) |
| Normalization at Week 52 in patients with abnormal | 9 | (5.8%) | 13 | (7.8%) |
| anti-dsDNA at baseline, n (%) | | | | |

C3^{a, c}

| | | | | |
|---|---|---|---|---|
| Abnormal C3 at baseline, n (%) | 137 | (37.4) | 130 | (36.1) |
| Mean (SD), U/mL | 0.70 | (0.14) | 0.69 | (0.15) |
| Change from baseline, mean (SD), U/mL | 0.04 | (0.16) | 0.13 | (0.18) |
| Normalization at Week 52 in patients with abnormal | 13 | (9.5) | 21 | (16.2) |
| C3 at baseline, n (%) | | | | |

C4^{a, d}

| | | | | |
|---|---|---|---|---|
| Abnormal C4 at baseline, n (%) | 85 | (23.2) | 84 | (23.3) |
| Mean (SD), U/mL | 0.07 | (0.02) | 0.07 | (0.02) |
| Change from baseline, mean (SD), U/mL | 0.02 | (0.04) | 0.02 | (0.03) |
| Normalization at Week 52 in patients with abnormal | 6 | (7.1) | 19 | (22.6) |
| C4 at baseline, n (%) | | | | |

anti-dsDNA, anti-double-stranded DNA; C3, complement 3; C4, complement 4; SD, standard deviation.
^{a}Only patients with baseline positive anti-dsDNA or low C3 or C4 are included in the summary statistics for the respective variables;
^{b}Anti-dsDNA antibody “positive” defined as a result of >15 U/mL;
^{c}Complement C3 “abnormal” levels defined as a result of <0.9 g/L;
^{d}Complement C4 “abnormal” levels defined as a result of <0.1 g/L.

### 12.4 Discussion

In this post hoc analysis of pooled data from the TULIP-1 and TULIP-2 trials, compared with placebo, anifrolumab treatment was associated with greater improvement in the most frequently affected organ domains (mucocutaneous, musculoskeletal, and immunologic) of patients with moderate to severe SLE. Anifrolumab treatment also resulted in greater improvements in skin disease and both swollen and tender joint counts, as well as in several less-prevalent domains, and in greater frequency of hematologic and serologic normalization compared with placebo.

Results of the TULIP-1 and TULIP-2 trials previously demonstrated that patients treated with anifrolumab had higher BICLA responder rates compared with patients receiving placebo. The present analyses also surprisingly demonstrate consistency between BILAG-2004 and SLEDAI-2K activity assessments for the most commonly affected individual organ domains. Baseline involvement of the mucocutaneous domain was present in ≥85% of patients as determined using BILAG-2004 and ≥90% using SLEDAI-2K, and comparable figures for the musculoskeletal domain were ≥85% and ≥95%, respectively. Using either BILAG-2004 or SLEDAI-2K organ domain responder assessments, greater improvement in mucocutaneous and musculoskeletal domains were observed with anifrolumab versus placebo, and improvements within these domains were comparable between indices.

Heat maps offer the advantage of visualizing both cohort- and patient-level responses across the entire study period, which has utility in a relapsing/remitting disease like SLE assessed with categorical values such as BILAG domain scores. The heat maps generated in this analysis illustrate that BILAG-2004 scores of patients with organ involvement at baseline varied during the study. This is expected owing to the clinical instability of SLE, which is characterized by intermittent periods of disease flare. Notwithstanding this, more frequent and earlier responses were observed with anifrolumab versus placebo across multiple domains. Very early separation in responses for laboratory domains, such as immunologic and hematologic, may reflect the role of IFN in disease activity in these organ systems or greater sensitivity to change in laboratory parameters compared with clinician-assessed disease activity.

In addition to analysis of BILAG-2004 and SLEDAI-2K mucocutaneous domains, the inventors used a validated skin-specific tool, CLASI, to assess skin disease in patients with SLE. The inventors assessed CLASI response in the subset of patients with CLASI-A ≥10 at baseline to focus on the patients with moderate to severe skin disease. Using all 3 skin disease measures, we observed a robust and early improvement of skin involvement in anifrolumab-treated patients compared with placebo.

Although ≥80% of patients with SLE report moderate to severe joint pain, there are no rigorously validated or widely accepted endpoints to assess musculoskeletal treatment response for patients with SLE, and there is no composite musculoskeletal outcome measure. In this study, the inventors analysed BILAG-2004 and SLEDAI-2K musculoskeletal domain responses, as well as changes in swollen and tender joint counts, each of which differ in their assessment of improvement. By all measures of musculoskeletal activity, patients treated with anifrolumab achieved greater improvements versus those receiving placebo. Both SLEDAI-2K and BILAG-2004 musculoskeletal domains include conditions other than arthritis. To concentrate on arthritis, the inventors focused on swollen and tender joint counts in patients with at least moderately severe arthritis at baseline, defined as either 26 or 28 swollen joints or ≥6 or 28 tender joints, similar to cut-offs used in enrolment in many trials of inflammatory joint disease. The inventors found that more patients treated with anifrolumab were able to achieve ≥50% reductions in baseline swollen and tender joint counts compared with those receiving placebo. The treatment effect was greater for swollen than for tender joints. Joint swelling in patients with SLE may be more likely to result from inflammation and is therefore potentially more responsive to immune-targeting treatments.

Serologic activity is indicative of immune system activation and is typically associated with SLE disease activity. More anifrolumab-treated patients were able to normalize anti-dsDNA antibodies and complement C3 and C4 levels compared with placebo-treated patients. These results suggest that the effects of anifrolumab on serologic markers are consistent with the greater improvements observed in those treated with anifrolumab compared with placebo in the SLEDAI-2K immunologic domain.

In conclusion, in pooled data from the phase 3 TULIP-1 and TULIP-2 trials, compared with placebo, anifrolumab treatment in patients with moderate to severe SLE was associated with improvements across organ systems, as measured by BILAG-2004 and SLEDAI-2K domain scores. In addition, more patients receiving anifrolumab compared with placebo had reductions in skin disease and swollen and tender joint counts. Together, these results provide evidence of the benefit of anifrolumab for reduction of disease activity across multiple organ domains in patients with active SLE.

### 13 Example 8: Efficacy of Anifrolumab in Serological Subgroups of Patients with SLE Participating in 2 Phase 3 Trials

### 13.1 Background

In the TULIP-2 and TULIP-1 trials of patients with SLE, the type I IFN receptor mAb anifrolumab resulted in higher BILAG-based Composite Lupus Assessment (BICLA) response rates vs placebo at Week 52. Subgroup analyses revealed concordant BICLA response rates across clinically distinct SLE subgroups, including disease severity and SLE therapies. The inventors compare BICLA response rates in serological subgroups (low complement, anti-dsDNA antibody positivity, or both).

### 13.2 Methods

TULIP-2 (NCT02446899) and TULIP-1 (NCT02446912) were phase 3, randomized, placebo-controlled, 52-week trials of intravenous anifrolumab every 4 weeks for 48 weeks in eligible patients who fulfilled the ACR criteria for SLE and had moderate to severe SLE despite standard therapy. BICLA response rates at Week 52 for anifrolumab vs placebo groups were compared across patient subgroups of baseline complement C3/C4 levels (low/normal) and anti-dsDNA antibody status (positive/negative).

### 13.3 Results

In TULIP-2 and TULIP-1, 180 patients in each trial received anifrolumab 300 mg, and 182 and 184 patients received placebo in TULIP-2 and TULIP-1, respectively. BICLA response rates in the overall anifrolumab groups were similar in TULIP-2 and TULIP-1 (47.8% and 47.1%, respectively), with treatment differences (A) favouring anifrolumab over placebo (A=16.3% and 17.0%, respectively) (FIG. 19). Anifrolumab response rates were higher in patients with baseline abnormal serologies vs those with normal serologies (range, 47.7%-53.0% vs 42.8%-47.9%), with the greatest anifrolumab response rate seen in the low C3/C4 subgroup (52.9% and 53.0% in TULIP-2 and TULIP-1, respectively). In contrast, placebo response rates were lower in serologically abnormal vs normal subgroups (range, 24.7%-31.6% vs 28.7%-35.8%). Anifrolumab and placebo subgroup response rates did not vary by more than ±5% from the overall population. These variations in response rates led to greater treatment differences favouring anifrolumab over placebo in serologically abnormal subgroups (range, A=16.1%-28.4%), with the largest difference seen for patients with low C3/C4 (A=26.9% and 28.4% in TULIP-2 and TULIP-1, respectively). In the subgroups with normal serology i.e., either normal C3/C4, no anti-dsDNA positivity, or both, treatment differences ranged from 7.5%-16.2%; however, treatment differences favoured anifrolumab vs placebo in all evaluated subgroups, regardless of serology.

### 13.4 CONCLUSIONS

BICLA response rates in clinically distinct subgroups of SLE were generally consistent with the overall TULIP-2 and TULIP-1 populations; however, patients with abnormal serologies had greater treatment effects than those with normal serologies.

### 14 EXAMPLE 9: SLE Treatment History and Anifrolumab Efficacy by Baseline Standard Therapies in Patients with Systemic Lupus Erythematosus from 2 Phase 3 Trials

### 14.1 Background

In the phase 3 TULIP-1 and TULIP-2 trials, anifrolumab, a type I IFN receptor mAb, improved disease activity versus placebo in patients who had moderate to severe SLE despite standard therapy with oral glucocorticoids (GCs), antimalarials, and/or immunosuppressants. The inventors investigated prior standard therapy use, and whether baseline standard therapy impacted anifrolumab efficacy in pooled data from TULIP-1 and TULIP-2.

### 14.2 Methods

TULIP-1 (NCT02446912) and TULIP-2 (NCT02446899) were 52-week trials of intravenous anifrolumab 300 mg or placebo every 4 weeks for 48 weeks, in which eligible patients fulfilled the ACR criteria for SLE. At screening, all patients had moderate to severe SLE (SLEDAI-2K ≥6, ≥1 A or ≥2 B BILAG-2004 organ domain scores, Physician's Global Assessment ≥1) and were required to be receiving ≥1 of the following: oral GCs, antimalarials, immunosuppressants (azathioprine, mizoribine, mycophenolate mofetil, mycophenolic acid, and/or methotrexate). Patients were divided into subgroups of SLE treatments at baseline. British Isles Lupus Assessment Group-based Combined Lupus Assessment (BICLA) response at Week 52 was compared across baseline SLE treatment subgroups using a stratified Cochran-Mantel-Haenszel approach.

### 14.3 Results

Overall, 726 patients received anifrolumab 300 mg (n=360) or placebo (n=366) in TULIP-1 and TULIP-2. Demographics and baseline disease characteristics were generally balanced between treatment groups. The median time from SLE diagnosis to randomization (prior to baseline) was 84.5 months, during which 89.5% of patients had received GCs, 84.3% had received antimalarials, and 68.0% had received immunosuppressants. Prior to baseline, all patients had received ≥1 SLE-related therapy, 34.3% had received 2 SLE-related therapies, and 57.3% of patients had received ≥3 SLE-related therapies. At baseline, patients were receiving GCs (82.0%), antimalarials (70.2%), and/or immunosuppressants (48.2%), with most patients receiving combinations of the three (Table 14-1). Anifrolumab 300 mg was associated with higher BICLA response rates versus placebo across all evaluated baseline SLE standard therapy subgroups, with positive treatment differences ranging from 6.9% (antimalarial+immunosuppressant) to 50.8% (immunosuppressant only) (FIG. 20); however, some groups had small sample sizes and the impact of dosage on efficacy was not investigated. Furthermore, positive treatment differences favouring anifrolumab 300 mg vs placebo were observed in patients who were receiving GCs+antimalarials+immunosuppressants at baseline (53.6% vs 32.2%; A=21.4%; 95% CI: 7.4-35.4), who were likely to have treatment-refractory disease.

TABLE 14-1
Background standard therapy regimens prior to and at baseline in TULIP-1 and TULIP-2

| | Prior to baseline^{a} | | | | At baseline^{b} | | | |
|---|---|---|---|---|---|---|---|---|
| | Anifrolumab | | | | Anifrolumab | | | |
| | 300 mg | | Placebo | | 300 mg | | Placebo | |
| SLE standard therapies | (n = 360) | | (n = 366) | | (n = 360) | | (n = 366) | |
| Any oral GC^{c} | 325 | (90.3) | 325 | (88.8) | 291 | (80.8) | 304 | (83.1) |
| Oral GC only | 28 | (7.8) | 21 | (5.7) | 56 | (15.6) | 38 | (10.4) |
| Oral GC + antimalarial | 297 | (82.5) | 304 | (83.1) | 235 | (65.3) | 266 | (72.7) |
| and/or immunosuppressant | | | | | | | | |
| Any antimalarials | 299 | (83.1) | 313 | (85.5) | 243 | (67.5) | 267 | (73.0) |
| Antimalarial only | 28 | (7.8) | 27 | (7.4) | 32 | (8.9) | 38 | (10.4) |
| Antimalarial + oral GC | 271 | (75.3) | 286 | (78.1) | 211 | (58.6) | 229 | (62.6) |
| and/or immunosuppressant | | | | | | | | |
| Any immunosuppressants | 248 | (68.9) | 246 | (67.2) | 173 | (48.1) | 177 | (48.4) |
| Azathioprine | 121 | (33.6) | 113 | (30.9) | 62 | (17.2) | 61 | (16.7) |
| Cyclophosphamide | 50 | (13.9) | 39 | (10.7) | — | | — | |
| Leflunomide | 9 | (2.5) | 9 | (2.5) | — | | — | |
| Methotrexate | 104 | (28.9) | 135 | (36.9) | 56 | (15.6) | 73 | (19.9) |
| Mizoribine | 9 | (2.5) | 11 | (3.0) | 4 | (1.1) | 3 | (0.8) |
| Mycophenolate^{d} | 82 | (22.8) | 79 | (21.6) | 54 | (15.0) | 45 | (12.3) |
| Tacrolimus | 18 | (5.0) | 23 | (6.3) | — | | — | |
| ≥2 different | 103 | (28.6) | 102 | (27.9) | 3 | (0.8) | 5 | (1.4) |
| immunosuppressants | | | | | | | | |

GC, glucocorticoid.
^{a}Includes any SLE standard therapies used since SLE diagnosis with start date prior to randomization;
^{b}Baseline is defined as the last measurement prior to randomization and investigational product dose administration on Day 1;
^{c}Prednisone or equivalent;
^{d}Mycophenolate or mycophenolic acid.

### 14.4 Conclusions

In 2 phase 3 trials, there were consistently higher BICLA response rates with anifrolumab 300 mg than with placebo, regardless of SLE standard therapy usage, including in patients with potentially more treatment-refractory SLE that required treatment with GCs, immunosuppressants, and antimalarials.

### 15 EXAMPLE 10: Injection Device

Anifrolumab is administered by an injection device [1] [9] such as a prefilled syringe (PFS) (FIG. 21A) or an autoinjector (AI) (FIG. 21B).

### 15.1 Autoinjector

Anifrolumab may be administered by an autoinjector [1]. The autoinjector is shown in exploded view (FIG. 22A) and in an assembled form (FIG. 22B). A label [4] is wrapped around and attached to the autoinjector [1] (FIG. 22C). The autoinjector has an autoinjector housing [3], cap and cap remover [2] and drive unit [5]. The liquid anifrolumab formulation unit dose [6] is contained in the autoinjector housing [3]. The unit dose [6] can be viewed through the viewing window [7].

### 15.2 Accessorized Pre-Frilled Syringe

Anifrolumab may be administered by accessorized pre-filled syringe (APFS) [8]. The APFS [8] includes the unit dose of anifrolumab [6] contained in a primary container [9] shown in an assembled state in FIG. 23A and in an exploded view in FIG. 23B. The primary container [9] has a plunger stopper [16]. The primary container has a nominal fill volume [17] of 0.8 ml but may contain slightly more than 0.8 ml. The remainder of the space in the primary container [9] is taken up by an air bubble [18]. The air bubble [18] may have a size of 3-5 mm, optionally, 4 mm. The primary container [9] has a defined stopper position [19].

The accessorized pre-filled syringe (APFS) primary container [9] is provided in a PFS assembly [8] including a needle guard [12], a finger flange [11] and a plunger rod [13] (FIG. 23C, FIG. 23D). A label [14] is provided with the primary container [9] in the PFS assembly [8]. The label [14] is wrapped around the syringe [9] in the label placement position [15].

### 15.3 Packaging

The injection device [1] [8] is provided in a kit [20] (FIG. 24). A label [4] [14] is provided with the APFS or autoinjector in the packaging. The label includes instruction for the use of the injection device [1], [8]. The packaging includes a tamper seal.

### REFERENCES

All publications mentioned in the specification are herein incorporated by reference.
(1) Pons-Estel, G. J.; Alarcón, G. S.; Scofield, L.; Reinlib, L.; Cooper, G. S. Understanding the Epidemiology and Progression of Systemic Lupus Erythematosus. Semin Arthritis Rheum 2010, 39 (4), 257-268. https://doi.org/10.1016/j.semarthrit.2008.10.007.
(2) McCauliffe, D. P. Cutaneous Lupus Erythematosus. Semin Cutan Med Surg 2001, 20 (1), 14-26. https://doi.org/10.1053/sder.2001.23091.
(3) Uva, L.; Miguel, D.; Pinheiro, C.; Freitas, J. P.; Marques Gomes, M.; Filipe, P. Cutaneous Manifestations of Systemic Lupus Erythematosus. Autoimmune Dis 2012, 2012, 834291. https://doi.org/10.1155/2012/834291.
(4) Zayat, A. S.; Md Yusof, M. Y.; Wakefield, R. J.; Conaghan, P. G.; Emery, P.; Vital, E. M. The Role of Ultrasound in Assessing Musculoskeletal Symptoms of Systemic Lupus Erythematosus: A Systematic Literature Review. Rheumatology (Oxford) 2016, 55 (3), 485-494. https://doi.org/10.1093/rheumatology/kev343.
(5) Sa, C.; E, A.; A, R.; D, I. Damage and mortality in a group of British patients with systemic lupus erythematosus followed up for over 10 years https://pubmed.ncbi.nlm.nih.gov/19359343/(accessed Feb. 8, 2021). https://doi.org/10.1093/rheumatology/kep062.
(6) Murimi-Worstell, I. B.; Lin, D. H.; Nab, H.; Kan, H. J.; Onasanya, O.; Tierce, J. C.; Wang, X.; Desta, B.; Alexander, G. C.; Hammond, E. R. Association between Organ Damage and Mortality in Systemic Lupus Erythematosus: A Systematic Review and Meta-Analysis. BMJ Open 2020, 10 (5), e031850. https://doi.org/10.1136/bmjopen-2019-031850.
(7) Doria, A.; Briani, C. Lupus: Improving Long-Term Prognosis. Lupus 2008, 17 (3), 166-170. https://doi.org/10.1177/0961203307087612.
(8) Petri, M. Long-Term Outcomes in Lupus. Am J Manag Care 2001, 7 (16 Suppl), S480-485.
(9) Zonana-Nacach, A.; Barr, S. G.; Magder, L. S.; Petri, M. Damage in Systemic Lupus Erythematosus and Its Association with Corticosteroids. Arthritis Rheum 2000, 43 (8), 1801-1808. https://doi.org/10.1002/1529-0131(200008) 43:8<1801::AID-ANR16≥3.0.CO; 2-O.
(10) Urowitz, M. B.; Bookman, A. A.; Koehler, B. E.; Gordon, D. A.; Smythe, H. A.; Ogryzlo, M. A. The Bimodal Mortality Pattern of Systemic Lupus Erythematosus. Am J Med 1976, 60 (2), 221-225. https://doi.org/1 0.1016/0002-9343(76) 90431-9.
(11) Patel, D. D.; Antoni, C.; Freedman, S. J.; Levesque, M. C.; Sundy, J. S. Phase 2 to Phase 3 Clinical Trial Transitions: Reasons for Success and Failure in Immunologic Diseases. Journal of Allergy and Clinical Immunology 2017, 140 (3), 685-687. https://doi.org/1 0.1016/j.jaci.2017.04.029.
(12) Dowden, H.; Munro, J. Trends in Clinical Success Rates and Therapeutic Focus. Nature Reviews Drug Discovery 2019, 18 (7), 495-496. https://doi.org/i 0.1038/d41573-019-00074-z.
(13) Eisenberg, R. WHY CAN'T WE FIND A NEW TREATMENT FOR SLE? JAutoimmun 2009, 32 (3-4), 223-230. https://doi.org/10.1016/j.jaut.2009.02.006.
(14) Isenberg, D. A.; Petri, M.; Kalunian, K.; Tanaka, Y.; Urowitz, M. B.; Hoffman, R. W.; Morgan-Cox, M.; likuni, N.; Silk, M.; Wallace, D. J. Efficacy and Safety of Subcutaneous Tabalumab in Patients with Systemic Lupus Erythematosus: Results from ILLUMINATE-1, a 52-Week, Phase III, Multicentre, Randomised, Double-Blind, Placebo-Controlled Study. Ann Rheum Dis 2016, 75 (2), 323-331. https://doi.org/10.1136/annrheumdis-2015-207653.
(15) Isenberg, D.; Merrill, J.; Hoffman, R.; Linnik, M.; Morgan-Cox, M.; Veenhuizen, M.; likuni, N.; Dickson, C.; Silk, M.; Wallace, D.; Dbrner, T. OP0184 Efficacy and Safety of Tabalumab in Patients with Systemic Lupus Erythematosus (SLE): Results from 2 Phase 3, 52-Week, Multicenter, Randomized, Placebo-Controlled Trials. Annals of the Rheumatic Diseases 2015, 74 (Suppl 2), 141-141. https://doi.org/10.1136/annrheumdis-2015-eular.1195.
(16) Furie, R. A.; Leon, G.; Thomas, M.; Petri, M. A.; Chu, A. D.; Hislop, C.; Martin, R. S.; Scheinberg, M. A.; PEARL-SC Study. A Phase 2, Randomised, Placebo-Controlled Clinical Trial of Blisibimod, an Inhibitor of B Cell Activating Factor, in Patients with Moderate-to-Severe Systemic Lupus Erythematosus, the PEARL-SC Study. Ann Rheum Dis 2015, 74 (9), 1667-1675. https://doi.org/10.1136/annrheumdis-2013-205144.
(17) Merrill, J. T.; Shanahan, W. R.; Scheinberg, M.; Kalunian, K. C.; Wofsy, D.; Martin, R. S. Phase III Trial Results with Blisibimod, a Selective Inhibitor of B-Cell Activating Factor, in Subjects with Systemic Lupus Erythematosus (SLE): Results from a Randomised, Double-Blind, Placebo-Controlled Trial. Annals of the Rheumatic Diseases 2018, 77 (6), 883-889. https://doi.org/10.1136/annrheumdis-2018-213032.
(18) Isenberg, D.; Gordon, C.; Licu, D.; Copt, S.; Rossi, C. P.; Wofsy, D. Efficacy and Safety of Atacicept for Prevention of Flares in Patients with Moderate-to-Severe Systemic Lupus Erythematosus (SLE): 52-Week Data (APRIL-SLE Randomised Trial). Ann Rheum Dis 2015, 74 (11), 2006-2015. https://doi.org/10.1136/annrheumdis-2013-205067.
(19) Alarcón-Segovia, D.; Tumlin, J. A.; Furie, R. A.; McKay, J. D.; Cardiel, M. H.; Strand, V.; Bagin, R. G.; Linnik, M. D.; Hepburn, B.; LJP 394 Investigator Consortium. LJP 394 for the Prevention of Renal Flare in Patients with Systemic Lupus Erythematosus: Results from a Randomized, Double-Blind, Placebo-Controlled Study. Arthritis Rheum 2003, 48 (2), 442-454. https://doi.org/1 0.1002/art.10763.
(20) Mahieu, M. A.; Strand, V.; Simon, L. S.; Lipsky, P. E.; Ramsey-Goldman, R. A Critical Review of Clinical Trials in Systemic Lupus Erythematosus. Lupus 2016, 25 (10), 1122-1140. https://doi.org/1 0.1177/0961203316652492.
(21) Rovin, B. H.; Furie, R.; Latinis, K.; Looney, R. J.; Fervenza, F. C.; Sanchez-Guerrero, J.; Maciuca, R.; Zhang, D.; Garg, J. P.; Brunetta, P.; Appel, G.; LUNAR Investigator Group. Efficacy and Safety of Rituximab in Patients with Active Proliferative Lupus Nephritis: The Lupus Nephritis Assessment with Rituximab Study. Arthritis Rheum 2012, 64 (4), 1215-1226. https://doi.org/10.1002/art.34359.
(22) Merrill, J. T.; Neuwelt, C. M.; Wallace, D. J.; Shanahan, J. C.; Latinis, K. M.; Oates, J. C.; Utset, T. O.; Gordon, C.; Isenberg, D. A.; Hsieh, H.-J.; Zhang, D.; Brunetta, P. G. Efficacy and Safety of Rituximab in Moderately-to-Severely Active Systemic Lupus Erythematosus: The Randomized, Double-Blind, Phase II/III Systemic Lupus Erythematosus Evaluation of Rituximab Trial. Arthritis Rheum 2010, 62 (1), 222-233. https://doi.org/1 0.1002/art.27233.
(23) Daikh, D. I.; Wofsy, D. Cutting Edge: Reversal of Murine Lupus Nephritis with CTLA4Ig and Cyclophosphamide. The Journal of Immunology 2001, 166 (5), 2913-2916. https://doi.org/1 0.4049/jimmunol.166.5.2913.
(24) Merrill, J. T.; Burgos-Vargas, R.; Westhovens, R.; Chalmers, A.; D'Cruz, D.; Wallace, D. J.; Bae, S. C.; Sigal, L.; Becker, J.-C.; Kelly, S.; Raghupathi, K.; Li, T.; Peng, Y.; Kinaszczuk, M.; Nash, P. The Efficacy and Safety of Abatacept in Patients with Non-Life-Threatening Manifestations of Systemic Lupus Erythematosus: Results of a Twelve-Month, Multicenter, Exploratory, Phase IIb, Randomized, Double-Blind, Placebo-Controlled Trial. Arthritis Rheum 2010, 62 (10), 3077-3087. https://doi.org/10.1002/art.27601.
(25) Strand, V.; Petri, M.; Kalunian, K.; Gordon, C.; Wallace, D. J.; Hobbs, K.; Kelley, L.; Kilgallen, B.; Wegener, W. A.; Goldenberg, D. M. Epratuzumab for Patients with Moderate to Severe Flaring SLE: Health-Related Quality of Life Outcomes and Corticosteroid Use in the Randomized Controlled ALLEVIATE Trials and Extension Study SL0006. Rheumatology (Oxford) 2014, 53 (3), 502-511. https://doi.org/1 0.1093/rheumatology/ket378.
(26) Wallace, D. J.; Kalunian, K.; Petri, M. A.; Strand, V.; Houssiau, F. A.; Pike, M.; Kilgallen, B.; Bongardt, S.; Barry, A.; Kelley, L.; Gordon, C. Efficacy and Safety of Epratuzumab in Patients with Moderate/Severe Active Systemic Lupus Erythematosus: Results from EMBLEM, a Phase IIb, Randomised, Double-Blind, Placebo-Controlled, Multicentre Study. Ann Rheum Dis 2014, 73 (1), 183-190. https://doi.org/10.1136/annrheumdis-2012-202760.
(27) Wallace, D. J.; Strand, V.; Merrill, J. T.; Popa, S.; Spindler, A. J.; Eimon, A.; Petri, M.; Smolen, J. S.; Wajdula, J.; Christensen, J.; Li, C.; Diehl, A.; Vincent, M. S.; Beebe, J.; Healey, P.; Sridharan, S. Efficacy and Safety of an Interleukin 6 Monoclonal Antibody for the Treatment of Systemic Lupus Erythematosus: A Phase II Dose-Ranging Randomised Controlled Trial. Annals of the Rheumatic Diseases 2017, 76 (3), 534-542. https://doi.org/1 0.1136/annrheumdis-2016-209668.
(28) Morand, E. F.; Furie, R.; Tanaka, Y.; Bruce, I. N.; Askanase, A. D.; Richez, C.; Bae, S.-C.; Brohawn, P. Z.; Pineda, L.; Berglind, A.; Tummala, R. Trial of Anifrolumab in Active Systemic Lupus Erythematosus. New England Journal of Medicine 2020, 382 (3), 211-221. https://doi.org/10.1056/NEJMoa1912196.
(29) Furie, R.; Khamashta, M.; Merrill, J. T.; Werth, V. P.; Kalunian, K.; Brohawn, P.; IIIei, G. G.; Drappa, J.; Wang, L.; Yoo, S.; Investigators, for the C. S. Anifrolumab, an Anti-Interferon-α Receptor Monoclonal Antibody, in Moderate-to-Severe Systemic Lupus Erythematosus. Arthritis & Rheumatology (Hoboken, N.j.) 2017, 69 (2), 376. https://doi.org/10.1002/art.39962.
(30) Furie, R. A.; Morand, E. F.; Bruce, I. N.; Manzi, S.; Kalunian, K. C.; Vital, E. M.; Ford, T. L.; Gupta, R.; Hiepe, F.; Santiago, M.; Brohawn, P. Z.; Berglind, A.; Tummala, R. Type I Interferon Inhibitor Anifrolumab in Active Systemic Lupus Erythematosus (TULIP-1): A Randomised, Controlled, Phase 3 Trial. The Lancet Rheumatology 2019, 1 (4), e208-e219. https://doi.org/i 0.1016/S2665-9913(19) 30076-1.
(31) Isenberg, D. A.; Merrill, J. T. Why, Why, Why de-Lupus (Does so Badly in Clinical Trials). Expert Rev Clin Immunol 2016, 12 (2), 95-98. https://doi.org/10.1586/1744666X.2016.1112270.
(32) Wallace, D. J. The Evolution of Drug Discovery in Systemic Lupus Erythematosus. Nat Rev Rheumatol 2015, 11 (10), 616-620. https://doi.org/10.1038/nrrheum.2015.86.
(33) Felten, R.; Sagez, F.; Gavand, P.-E.; Martin, T.; Korganow, A.-S.; Sordet, C.; Javier, R.-M.; Soulas-Sprauel, P.; Rivibre, M.; Scher, F.; Poindron, V.; Guffroy, A.; Arnaud, L. 10 Most Important Contemporary Challenges in the Management of SLE. Lupus Science & Medicine 2019, 6 (1), e000303. https://doi.org/1 0.1136/lupus-2018-000303.
(34) Yao, Y.; Higgs, B. W.; Richman, L.; White, B.; Jallal, B. Use of Type I Interferon-Inducible MRNAs as Pharmacodynamic Markers and Potential Diagnostic Markers in Trials with Sifalimumab, an Anti-IFNa Antibody, in Systemic Lupus Erythematosus. Arthritis Res Ther 2010, 12 (Suppl 1), S6. https://doi.org/10.1186/ar2887.
(35) Albrecht, J.; Taylor, L.; Berlin, J. A.; Dulay, S.; Ang, G.; Fakharzadeh, S.; Kantor, J.; Kim, E.; Militello, G.; McGinnis, K.; Richardson, S.; Treat, J.; Vittorio, C.; Van Voorhees, A.; Werth, V. P. The CLASI (Cutaneous Lupus Erythematosus Disease Area and Severity Index): An Outcome Instrument for Cutaneous Lupus Erythematosus. J Invest Dermatol 2005, 125 (5), 889-894. https://doi.org/10.1111/j.0022-202X.2005.23889.x.
(36) Klein, R. S.; Morganroth, P. A.; Werth, V. P. Cutaneous Lupus and the CLASI Instrument. Rheum Dis Clin North Am 2010, 36 (1), 33-51. https://doi.org/10.1016/j.rdc.2009.12.001.
(37) Touma, Z.; Urowitz, M.; Gladman, D. SLEDAI-2K for a 30-Day Window. Lupus 2010, 19 (1), 49-51. https://doi.org/1 0.1177/0961203309346505.
(38) Ferguson, G. T.; Mansur, A. H.; Jacobs, J. S.; Hebert, J.; Clawson, C.; Tao, W.; Wu, Y.; Goldman, M. Assessment of an Accessorized Pre-Filled Syringe for Home-Administered Benralizumab in Severe Asthma. J Asthma Allergy 2018, 11, 63-72. https://doi.org/10.2147/JAA.S157762.
(39) Interferon-Inducible Gene Expression Kit As a Potential Diagnostic Test for Anifrolumab: Analytical Validation for Use in Clinical Trials. ACR Meeting Abstracts.

1. A method of treating or preventing mucocutaneous, musculoskeletal and/or renal disease in an systemic lupus erythematosus (SLE) patient in need thereof, the method comprising administering a therapeutically effective amount of a type I IFN receptor (IFNAR1) inhibitor to the patient, wherein the method treats mucocutaneous, musculoskeletal and/or renal disease in the patient.

2. The method of claim 1, wherein the method treats mucocutaneous, musculoskeletal and renal disease in the patient.

3. The method of claim 1 or 2, wherein the method reduces the mucocutaneous, musculoskeletal and/or renal flare rate in the patient relative to pre-treatment mucocutaneous, musculoskeletal and/or renal flare rate respectively.

4. The method of any preceding claim, wherein the method improves the patient's BILAG-2004 mucocutaneous, renal and/or musculoskeletal organ domain score.

5. The method of any preceding claim, wherein the method improves the patient's SLEDAI-2K mucocutaneous and/or musculoskeletal organ domain score.

6. The method of any preceding claim, wherein the method treats cardiorespiratory disease in the patient, optionally wherein the method improves the patient's BILAG-2004 cardiorespiratory organ domain score.

7. The method of any preceding claim, wherein the method treats constitutional disease in the patient, optionally wherein the method improves the patient's BILAG-2004 constitutional organ domain score.

8. The method of any preceding claim, wherein the method treats vascular, hematologic, renal and/or cardiorespiratory disease in the patient, optionally wherein the method improves the patient's SLEDAI-2K vascular, hematologic, renal and/or cardiorespiratory disease organ domain score.

9. The method of any preceding claim, wherein the method treats rash in the patient.

10. The method of claim 9, wherein there is a ≥50% improvement in rash in the subject from pre-treatment levels of rash, optionally wherein the improvement is defined by Cutaneous Lupus Erythematosus Disease Area and Severity Index (CLASI).

11. The method of claim 10, wherein the method resolves rash in the patient, optionally wherein the method completely resolves SLEDAI-2K-defined rash in the patient.

12. The method of any preceding claim, wherein the method treats or prevent arthritis in the patient.

13. The method of claim 12, wherein the method completely resolves arthritis in the patient, optionally wherein the method complete resolves SLEDAI-2K-defined arthritis in the patient.

14. The method of any preceding claim, wherein the method leads to a ≥50% improvement in swollen and tender joint count in the patient compared to the pre-treatment swollen and tender joint count in the patient, optionally wherein the patient had ≥6 swollen and tender joint count pre-treatment.

15. The method any preceding claim, wherein the method comprises a method of treating or preventing renal disease in the patient, wherein the method treats or prevents renal disease in the patient.

16. The method of claim 15, wherein the patient has a 24-hour UPCR ≥0.5 mg/mg pre-treatment, and wherein the method improves the subject's 24-hour UPCR to 50.5 mg/mg.

17. A method of treating SLE in a patient thereof, the method comprising administering a therapeutically effective amount of a type I IFN receptor (IFNAR1) inhibitor to the patient, wherein the patient has a baseline CLASI-A ≥10, wherein treatment reduces the patient's CLASI-A ≥50%.

18. The method of claim 17, wherein the treatment reduces the patient's CLASI-A by at least week 12 of treatment.

19. The method of claim 17 or 18, wherein the reduction in the patient's CLASI-A is maintained for at least 4, 8, 12, 16, 20, 24, 28, 32, 36 or 40 weeks.

20. A method of treating a systemic lupus erythematosus (SLE) patient in need thereof, the method comprising administering a therapeutically effective amount of a type I IFN receptor (IFNAR1) inhibitor to the patient, wherein them subject has low complement at baseline compared to a healthy subject, wherein the method reduces SLE disease activity in the patient.

21. The method of claim 20, where the patient has low C3 and/or C4 complement at baseline compared to a healthy subject.

22. A method of treating a systemic lupus erythematosus (SLE) patient in need thereof, the method comprising administering a therapeutically effective amount of a type I IFN receptor (IFNAR1) inhibitor to the patient, wherein them patient has treatment-refractory SLE, and wherein the method reduces SLE disease activity in the patient.

23. The method of claim 22, wherein the patient has previously received prior treatment with glucocorticoids, antimalarials and/or immunosuppressants.

24. The method of claim 22 or 23, wherein pre-treatment the patient has a SLEDAI-2K score of ≥6, ≥1 A and/or a ≥2 B BILAG-2004 organ domain score, and/or a Physician's Global Assessment of ≥1.

25. The method of any of claims 22 to 24, wherein the patient has received prior treatment with azathioprine, mizoribine, mycophenolate mofetil, mycophenolic acid, and/or methotrexate.

26. The method of any of claims 20 to 25, wherein reducing SLE disease activity in the patient comprises a BILAG-Based Composite Lupus Assessment (BICLA) response.

27. The method of any preceding claim, wherein the patient has moderate to severe SLE.

28. The method of any preceding claim, wherein the method has been demonstrated in a phase III clinical trial.

29. The method of any preceding claim, wherein the type I IFN receptor inhibitor is anifrolumab or a functional variant thereof.

30. The method of claim 29, wherein the method comprises administering a fixed dose of anifrolumab.

31. The method of claim 30, wherein the method comprises administering about 300 mg to about 1000 mg of anifrolumab.

32. The method of claim 31, comprising administering about 300 mg anifrolumab.

33. The method of claim 31, comprising administering anifrolumab or the functional variant thereof at a dose of 300-1000 mg every four weeks (Q4W),

34. The method of claim 33, wherein anifrolumab or the functional variant thereof is administered intravenously.

35. The method of claim 31, comprising administering anifrolumab or the functional variant thereof to the patient at a dose of 120 mg every week, optionally wherein anifrolumab or the functional variant thereof is administered subcutaneously.

36. The method of any of the preceding claims, the method comprising steroid sparing in the subject, wherein the dose of the steroid administered to the subject is tapered from a pre-sparing dose at baseline to a post-sparing dose.

37. The method of claim 36, wherein the post-sparing dose is 57.5 mg/day prednisone or prednisone equivalent dose.

38. The method of any of claim 36 or 37, wherein the pre-sparing dose is 10 mg/day or prednisone equivalent dose.

39. The method of any of claims 36-38, wherein the steroid comprises a glucocorticoid.

40. The method of claim 39, wherein the steroid comprises an oral glucocorticoid.

41. The method of any of claims 36-40, wherein the steroid is hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof.

42. The method of any of claims 36-40, wherein the steroid comprises prednisone.

43. The method of any preceding claim, wherein the patient is a type I interferon stimulated gene signature (IFNGS)-test high patient pre-treatment.

44. The method of any preceding claim, comprising identifying the subject as IFNGS-test high patient before administration of the IFNAR1 inhibitor.

45. A pharmaceutical composition for use in any of the methods of claims 1-44.

46. An injection device comprising the pharmaceutical composition of claim 45.

47. The injection device of claim 46, wherein the injection device is a pre-filled syringe (PFS).

48. The injection device of claim 47, wherein the injection device is an accessorized pre-filed syringe (AFPS).

49. The injection device of claim 47, wherein the injection device is an auto-injector.

50. A kit comprising the injection device of any of claims 46-49, and instructions for use.

51. The kit of claim 50, wherein the instructions for use comprise instructions for subcutaneous administration of the pharmaceutical composition or unit dose to the patient.

52. The kit of claim 50 or 51, wherein the instructions for use specify that the injection device, unit dose and/or pharmaceutical composition are for use in the treatment of SLE.

53. The kit of any of claims 50-52, comprising packaging, wherein the packaging is adapted to hold the injection device and the instructions for use.

54. The kit of any of claims 50-53, wherein the instructions for use are attached to the injection device.
